SEQUENCE LISTING (15 sequences)

SEQ ID NO: 1 - DNA, 1953 bp, Artificial Sequence
  Synthetic primer
atgggatctgtgcaaagcaacctccaagctggagctgctgctgccagctgcatctccccaaagtactacatgatcttcactccttgcaagctctaccacctctgttgtagggagtctgagatcaacatgcacagcaaggttaccatcatttgcatcaggttcctcttttggttcctcctcctctgcatgcttattggtaagagccacactgaggatgacatcatcattgccaccaagaatggtaaggttaggggtatgaacctcacagtttttggtggtactgttacagccttccttggtattccttatgcccaaccacctcttggtagacttaggttcaagaagccacaaagcctcaccaagtggtctgacatttggaatgccaccaagtatgccaactcctgttgtcaaaacattgaccaatccttcccaggatttcatggatctgagatgtggaacccaaacactgacctctctgaggattgtctttaccttaatgtgtggatcccagccccaaagcctaagaatgccactgttctcatttggatctatggtggtggtttccaaactggaacctcctctctccatgtttatgatggaaagttcttggctagagttgagagagttattgtggtgagcatgaactatagggtgggtgccttgggattcttggccctcccaggaaatcctgaggccccaggtaatatgggtctttttgaccaacaattggctcttcaatgggttcagaagaacattgctgcctttggtggaaaccctaagtctgttaccctctttggagagtctgctggagctgcttctgttagccttcacttgctttctcctggaagccactccttgttcactagagccattctccaatctggatccttcaatgctccttgggctgtgacatctctttatgaggctaggaatagaacattgaaccttgctaagttgactggttgctctagagagaatgagactgagatcatcaagtgtcttagaaacaaggacccacaagagattcttttgaatgaggcctttgttgttccttatggaacccctttgtctgtgaactttggtcctacagtggatggtgatttcctcactgacatgccagacatcttgcttgagcttggacaattcaagaagacccaaattttggtgggtgttaacaaggatgagggtacagctttccttgtgtatggcgcgcctggttttagcaaggacaacaactccatcatcactagaaaggagttccaagagggtctcaagatcttcttcccaggagtgtctgagtttggaaaggagtccatccttttccattacacagattgggttgatgaccaaagacctgagaactatagggaggccttgggtgatgttgttggagattacaacttcatttgccctgccttggagttcaccaagaagttctctgagtggggaaataatgccttcttctactactttgagcataggtcctccaagctcccttggccagagtggatgggagtgatgcatggttatgagattgagtttgtttttggtttgcctcttgagagaagagataactacacaaaggctgaggagatcttgagcagatccattgtgaagaggtgggccaactttgccaagtatggtaatccaaatgagactcaaaacaatagcacaagctggcctgtgttcaagagcactgagcaaaagtacctcaccttgaacacagagtccacaaggattatgaccaagttgagggctcaacaatgtaggttttggacatccttcttcccaaaggtgttggagatgacaggaaatatcgatgaggctgagtgggagtggaaggctggattccataggtggaacaactacatgatggattggaagaaccaattcaatgattacactagcaagaaggagagctgtgtgggtctctctgagaaggatgaactctag

SEQ ID NO: 2 - protein, 650 aa, Artificial Sequence
  Synthetic peptide
MGSVQSNLQAGAAAASCISPKYYMIFTPCKLYHLCCRESEINMHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLSEKDEL

SEQ ID NO: 3 - DNA, 1821 bp, Artificial Sequence
  Synthetic primer
atggcgaacaaacacttgtccctctccctcttcctcgtcctccttggcctgtcggccagcttggcctccggagccatggaggatgacatcatcattgccaccaagaatggtaaggttaggggtatgaacctcacagtttttggtggtactgttacagccttccttggtattccttatgcccaaccacctcttggtagacttaggttcaagaagccacaaagcctcaccaagtggtctgacatttggaatgccaccaagtatgccaactcctgttgtcaaaacattgaccaatccttcccaggatttcatggatctgagatgtggaacccaaacactgacctctctgaggattgtctttaccttaatgtgtggatcccagccccaaagcctaagaatgccactgttctcatttggatctatggtggtggtttccaaactggaacctcctctctccatgtttatgatggaaagttcttggctagagttgagagagttattgtggtgagcatgaactatagggtgggtgccttgggattcttggccctcccaggaaatcctgaggccccaggtaatatgggtctttttgaccaacaattggctcttcaatgggttcagaagaacattgctgcctttggtggaaaccctaagtctgttaccctctttggagagtctgctggagctgcttctgttagccttcacttgctttctcctggaagccactccttgttcactagagccattctccaatctggatccttcaatgctccttgggctgtgacatctctttatgaggctaggaatagaacattgaaccttgctaagttgactggttgctctagagagaatgagactgagatcatcaagtgtcttagaaacaaggacccacaagagattcttttgaatgaggcctttgttgttccttatggaacccctttgtctgtgaactttggtcctacagtggatggtgatttcctcactgacatgccagacatcttgcttgagcttggacaattcaagaagacccaaattttggtgggtgttaacaaggatgagggtacagctttccttgtgtatggcgcgcctggttttagcaaggacaacaactccatcatcactagaaaggagttccaagagggtctcaagatcttcttcccaggagtgtctgagtttggaaaggagtccatccttttccattacacagattgggttgatgaccaaagacctgagaactatagggaggccttgggtgatgttgttggagattacaacttcatttgccctgccttggagttcaccaagaagttctctgagtggggaaataatgccttcttctactactttgagcataggtcctccaagctcccttggccagagtggatgggagtgatgcatggttatgagattgagtttgtttttggtttgcctcttgagagaagagataactacacaaaggctgaggagatcttgagcagatccattgtgaagaggtgggccaactttgccaagtatggtaatccaaatgagactcaaaacaatagcacaagctggcctgtgttcaagagcactgagcaaaagtacctcaccttgaacacagagtccacaaggattatgaccaagttgagggctcaacaatgtaggttttggacatccttcttcccaaaggtgttggagatgacaggaaatatcgatgaggctgagtgggagtggaaggctggattccataggtggaacaactacatgatggattggaagaaccaattcaatgattacactagcaagaaggagagctgtgtgggtctctctgagaaggatgaactctag

SEQ ID NO: 4 - protein, 605 aa, Artificial Sequence
  Synthetic peptide
MANKHLSLSLFLVLLGLSASLASGAMEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLSEKDEL

SEQ ID NO: 5 - DNA, 1821 bp, Artificial Sequence
  Synthetic primer
atggcgaacaaacacttgtccctctccctcttcctcgtcctccttggcctgtcggccagcttggcctccggagccatggaggatgacatcatcattgccaccaagaatggtaaggttaggggtatgaacctcacagtttttggtggtactgttacagccttccttggtattccttatgcccaaccacctcttggtagacttaggttcaagaagccacaaagcctcaccaagtggtctgacatttggaatgccaccaagtatgccaactcctgttgtcaaaacattgaccaatccttcccaggatttcatggatctgagatgtggaacccaaacactgacctctctgaggattgtctttaccttaatgtgtggatcccagccccaaagcctaagaatgccactgttctcatttggatctatggtggtggtttccaaactggaacctcctctctccatgtttatgatggaaagttcttggctagagttgagagagttattgtggtgagcatgaactatagggtgggtgccttgggattcttggccctcccaggaaatcctgaggccccaggtaatatgggtctttttgaccaacaattggctcttcaatgggttcagaagaacattgctgcctttggtggaaaccctaagtctgttaccctctttggagagtctgctggagctgcttctgttagccttcacttgctttctcctggaagccactccttgttcactagagccattctccaatctggatccttcaatgctccttgggctgtgacatctctttatgaggctaggaatagaacattgaaccttgctaagttgactggttgctctagagagaatgagactgagatcatcaagtgtcttagaaacaaggacccacaagagattcttttgaatgaggcctttgttgttccttatggaacccctttgtctgtgaactttggtcctacagtggatggtgatttcctcactgacatgccagacatcttgcttgagcttggacaattcaagaagacccaaattttggtgggtgttaacaaggatgagggtacagctttccttgtgtatggcgcgcctggttttagcaaggacaacaactccatcatcactagaaaggagttccaagagggtctcaagatcttcttcccaggagtgtctgagtttggaaaggagtccatccttttccattacacagattgggttgatgaccaaagacctgagaactatagggaggccttgggtgatgttgttggagattacaacttcatttgccctgccttggagttcaccaagaagttctctgagtggggaaataatgccttcttctactactttgagcataggtcctccaagctcccttggccagagtggatgggagtgatgcatggttatgagattgagtttgtttttggtttgcctcttgagagaagagataactacacaaaggctgaggagatcttgagcagatccattgtgaagaggtgggccaactttgccaagtatggtaatccaaatgagactcaaaacaatagcacaagctggcctgtgttcaagagcactgagcaaaagtacctcaccttgaacacagagtccacaaggattatgaccaagttgagggctcaacaatgtaggttttggacatccttcttcccaaaggtgttggagatgacaggaaatatcgatgaggctgagtgggagtggaaggctggattccataggtggaacaactacatgatggattggaagaaccaattcaatgattacactagcaagaaggagagctgtgtgggtctccatcaccatcaccatcactag

SEQ ID NO: 6 - protein, 605 aa, Artificial Sequence
  Synthetic peptide
MANKHLSLSLFLVLLGLSASLASGAMEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLHHHHHH

SEQ ID NO: 7 - DNA, 1821 bp, Artificial Sequence
  Synthetic primer
atggcgaacaaacacttgtccctctccctcttcctcgtcctccttggcctgtcggccagcttggcctccggagccatggaggatgacatcatcattgccaccaagaatggtaaggttaggggtatgaacctcacagtttttggtggtactgttacagccttccttggtattccttatgcccaaccacctcttggtagacttaggttcaagaagccacaaagcctcaccaagtggtctgacatttggaatgccaccaagtatgccaactcctgttgtcaaaacattgaccaatccttcccaggatttcatggatctgagatgtggaacccaaacactgacctctctgaggattgtctttaccttaatgtgtggatcccagccccaaagcctaagaatgccactgttctcatttggatctatggtggtggtttccaaactggaacctcctctctccatgtttatgatggaaagttcttggctagagttgagagagttattgtggtgagcatgaactatagggtgggtgccttgggattcttggccctcccaggaaatcctgaggccccaggtaatatgggtctttttgaccaacaattggctcttcaatgggttcagaagaacattgctgcctttggtggaaaccctaagtctgttaccctctttggagagtcttctggagctgcttctgttagccttcacttgctttctcctggaagccactccttgttcactagagccattctccaatctggttccgctaatgctccttgggctgtgacatctctttatgaggctaggaatagaacattgaaccttgctaagttgactggttgctctagagagaatgagactgagatcatcaagtgtcttagaaacaaggacccacaagagattcttttgaatgaggcctttgttgttccttatggaactcctttgggagtgaactttggtcctacagtggatggtgatttcctcactgacatgccagacatcttgcttgagcttggacaattcaagaagacccaaattttggtgggtgttaacaaggatgagggtacatggttccttgtgtctggagcgcctggttttagcaaggacaacaactccatcatcactagaaaggagttccaagagggtctcaagatcttcttcccaggagtgtctgagtttggaaaggagtccatccttttccattacacagattgggttgatgaccaaagacctgagaactatagggaggccttgggtgatgttgttggagattacaacttcatttgccctgccttggagttcaccaagaagttctctgagtggggaaataatgccttcttctactactttgagcataggtcctccaagctcccttggccagagtggatgggagtgatgcatggttatgagattgagtttgtttttggtttgcctcttgagagaagagataactacacaaaggctgaggagatcttgagcagatccattgtgaagaggtgggccaactttgccaagtatggtaatccaaatgagactcaaaacaatagcacaagctggcctgtgttcaagagcactgagcaaaagtacctcaccttgaacacagagtccacaaggattatgaccaagttgagggctcaacaatgtaggttttggacatccttcttcccaaaggtgttggagatgacaggaaatatcgatgaggctgagtgggagtggaaggctggattccataggtggaacaactacatgatggattggaagaaccaattcaatgattacactagcaagaaggagagctgtgtgggtctccatcaccatcaccatcactag

SEQ ID NO: 8 - protein, 606 aa, Artificial Sequence
  Synthetic peptide
MANKHLSLSLFLVLLGLSASLASGAMEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVSGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLHHHHHH

SEQ ID NO: 9 - DNA, 1821 bp, Artificial Sequence; identical to SEQ ID NO: 7
  Synthetic primer

SEQ ID NO: 10 - protein, 606 aa, Artificial Sequence
  Synthetic peptide
MANKHLSLSLFLVLLGLSASLASGAMEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLHHHHHH

SEQ ID NO: 11 - DNA, 1953 bp, Artificial Sequence; identical to SEQ ID NO: 1
  Synthetic primer

SEQ ID NO: 12 - protein, 650 aa, Artificial Sequence; identical to SEQ ID NO: 2
  Synthetic peptide

SEQ ID NO: 13 - DNA, 1842 bp, Artificial Sequence
  Synthetic primer
atgatcgttctttctgttggttccgcttcttcatctcctatcgtcgttgtcttttccgtggcacttcttctcttctacttctctgaaacttccctaggtgaggatgacatcatcattgccaccaagaatggtaaggttaggggtatgaacctcacagtttttggtggtactgttacagccttccttggtattccttatgcccaaccacctcttggtagacttaggttcaagaagccacaaagcctcaccaagtggtctgacatttggaatgccaccaagtatgccaactcctgttgtcaaaacattgaccaatccttcccaggatttcatggatctgagatgtggaacccaaacactgacctctctgaggattgtctttaccttaatgtgtggatcccagccccaaagcctaagaatgccactgttctcatttggatctatggtggtggtttccaaactggaacctcctctctccatgtttatgatggaaagttcttggctagagttgagagagttattgtggtgagcatgaactatagggtgggtgccttgggattcttggccctcccaggaaatcctgaggccccaggtaatatgggtctttttgaccaacaattggctcttcaatgggttcagaagaacattgctgcctttggtggaaaccctaagtctgttaccctctttggagagtctgctggagctgcttctgttagccttcacttgctttctcctggaagccactccttgttcactagagccattctccaatctggatccttcaatgctccttgggctgtgacatctctttatgaggctaggaatagaacattgaaccttgctaagttgactggttgctctagagagaatgagactgagatcatcaagtgtcttagaaacaaggacccacaagagattcttttgaatgaggcctttgttgttccttacggaactcctttgtctgtgaactttggtcctacagtggatggtgatttcctcactgacatgccagacatcttgcttgagcttggacaattcaagaagacccaaattttggtgggtgttaacaaggatgagggtacagctttccttgtgtatggcgcgcctggttttagcaaggacaacaactccatcatcactagaaaggagttccaagagggtctcaagatcttcttcccaggagtgtctgagtttggaaaggagtccatccttttccattacacagattgggttgatgaccaaagacctgagaactatagggaggccttgggtgatgttgttggagattacaacttcatttgccctgccttggagttcaccaagaagttctctgagtggggaaataatgccttcttctactactttgagcataggtcctccaagctcccttggccagagtggatgggagtgatgcatggttatgagattgagtttgtttttggtttgcctcttgagagaagagataactacacaaaggctgaggagatcttgagcagatccattgtgaagaggtgggccaactttgccaagtatggtaatccaaatgagactcaaaacaatagcacaagctggcctgtgttcaagagcactgagcaaaagtacctcaccttgaacacagagtccacaaggattatgaccaagttgagggctcaacaatgtaggttttggacatccttcttcccaaaggtgttggagatgacaggaaatatcgatgaggctgagtgggagtggaaggctggattccataggtggaacaactacatgatggattggaagaaccaattcaatgattacactagcaagaaggagagctgtgtgggtctccatcaccatcaccatcactag

SEQ ID NO: 14 - protein, 613 aa, Artificial Sequence
  Synthetic peptide
MIVLSVGSASSSPIVVVFSVALLLFYFSETSLGEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGLHHHHHH

SEQ ID NO: 15 - DNA, 1596 bp, Artificial sequence
  Synthetic sequence
atggaggatgacatcatcattgccaccaagaatggtaaggttaggggtatgaacctcacagtttttggtggtactgttacagccttccttggtattccttatgcccaaccacctcttggtagacttaggttcaagaagccacaaagcctcaccaagtggtctgacatttggaatgccaccaagtatgccaactcctgttgtcaaaacattgaccaatccttcccaggatttcatggatctgagatgtggaacccaaacactgacctctctgaggattgtctttaccttaatgtgtggatcccagccccaaagcctaagaatgccactgttctcatttggatctatggtggtggtttccaaactggaacctcctctctccatgtttatgatggaaagttcttggctagagttgagagagttattgtggtgagcatgaactatagggtgggtgccttgggattcttggccctcccaggaaatcctgaggccccaggtaatatgggtctttttgaccaacaattggctcttcaatgggttcagaagaacattgctgcctttggtggaaaccctaagtctgttaccctctttggagagtctgctggagctgcttctgttagccttcacttgctttctcctggaagccactccttgttcactagagccattctccaatctggatccttcaatgctccttgggctgtgacatctctttatgaggctaggaatagaacattgaaccttgctaagttgactggttgctctagagagaatgagactgagatcatcaagtgtcttagaaacaaggacccacaagagattcttttgaatgaggcctttgttgttccttatggaacccctttgtctgtgaactttggtcctacagtggatggtgatttcctcactgacatgccagacatcttgcttgagcttggacaattcaagaagacccaaattttggtgggtgttaacaaggatgagggtacagctttccttgtgtatggcgcgcctggttttagcaaggacaacaactccatcatcactagaaaggagttccaagagggtctcaagatcttcttcccaggagtgtctgagtttggaaaggagtccatccttttccattacacagattgggttgatgaccaaagacctgagaactatagggaggccttgggtgatgttgttggagattacaacttcatttgccctgccttggagttcaccaagaagttctctgagtggggaaataatgccttcttctactactttgagcataggtcctccaagctcccttggccagagtggatgggagtgatgcatggttatgagattgagtttgtttttggtttgcctcttgagagaagagataactacacaaaggctgaggagatcttgagcagatccattgtgaagaggtgggccaactttgccaagtatggtaatccaaatgagactcaaaacaatagcacaagctggcctgtgttcaagagcactgagcaaaagtacctcaccttgaacacagagtccacaaggattatgaccaagttgagggctcaacaatgtaggttttggacatcctctgagaaggatgaactctag

(19) United States
(12) Patent Application Publication
(10) Pub. No.: US 2014/0248684 A1
(43) Pub. Date: Sep. 4, 2014

(54) COMPOSITIONS AND METHODS FOR THE PRODUCTION AND USE OF HUMAN CHOLINESTERASES

(71) Applicants: Tsafrir Mor, Tempe, AZ (US); Latha Kannan, Gilbert, AZ (US); Katherine E. Larrimore, Tempe, AZ (US)

(72) Inventors: Tsafrir Mor, Tempe, AZ (US); Latha Kannan, Gilbert, AZ (US); Katherine E. Larrimore, Tempe, AZ (US)

(73) Assignee: ARIZONA BOARD OF REGENTS FOR AND ON BEHALF OF ARIZONA STATE UNIVERSITY, Scottsdale, AZ (US)

(21) Appl. No.: 14/344,974

(22) PCT Filed: Sep. 17, 2012
(86) PCT No.: PCT/US2012/055781

§ 371 Date: Mar. 14, 2014

Related U.S. Application Data
(60) Provisional application No. 61/535,528, filed on Sep. 16, 2011.

(51) Int. Cl. C12N 9/18; C12N 15/82

(52) U.S. Cl. CPC C12N 9/18; C12N 15/8257
USPC 435/197; 435/320.1

(57) ABSTRACT

In some aspects, the present invention relates to compositions and methods for the production of human cholinesterases. More particularly, it relates to methods for the production of human cholinesterases using transient expression and vectors for producing the same. In one aspect, the present invention relates to a plant viral vector encoding a plant codon-optimized DNA sequence that results in accumulation of the cholinesterase in a plant leaf at levels greater than 20 mg, and in some embodiments greater than 200 mg, of the enzyme per kilogram of the plant leaf.

This application claims the benefit of priority to U.S. Provisional Patent Application Ser. No. 61/535,528, filed Sep. 16, 2011, hereby incorporated by reference in its entirety.

The invention was made with government support under Grant No. P1 DA031340 awarded by the National Institute for Drug Abuse Program awarded to the Mayo Clinic and subcontracted to Arizona State University. The government has certain rights in this invention.

### BACKGROUND OF THE INVENTION

I. Field of the Invention

This invention relates to compositions and methods for the production human cholinesterases. More particularly, it relates to methods for the production of human cholinesterases using transient expression and vectors for producing the same.

II. Description of the Related Art

Organophosphorus (OP) compounds are highly toxic inhibitors of serine hydrolases. Although first explored as insecticides, the extreme toxicity of OPs toward mammals prompted their development as chemical warfare (CW) agents and the first military grade OP “nerve gases”, tabun, sarin and soman, were synthesized in Nazi Germany immediately prior to- and during World War II. The cold war era saw the unfortunate spread of the technology and the development of yet more toxic compounds such as VX, Russian-VX and cyclosarin. In fact, CW “nerve agents” (NAs) are relatively easy to produce, store and weaponize and their use by terrorists and rogue governments (exemplified by the Tokyo subway sarin attack by Aum Shinrikyo in 1995) pose a major threat to civilians and military personnel in the present global political climate. Thus, the use of OP NAs as a means of terror and nonconventional warfare aggrandizes the peace-time concern of accidental and environmental exposure to OP pesticides (Greenfield et al., 2002; Lee, 2003).

Bioscavenging of organophosphate (OP) by human proteins is emerging as a promising medical intervention for prophylaxis and post-exposure treatment against chemical warfare nerve agents. The best-studied bioscavengers (BSCs) to date, meeting considerable success in pre-clinical research, are human cholinesterases (ChEs) (Ashani, 2000; Doctor and Saxena, 2005). However, ChEs, which are highly efficient in binding and sequestering OPs, are also inactivated by the toxins and therefore operate as stoichiometric rather than catalytic BSCs. This necessitates the availability of large quantities of enzymes. In the near term, outdated human plasma can be a first generation source of one such enzyme, butyrylcholinesterase (BChE), that may be used in clinical trials to validate its safety and effectiveness in biodefense. In the longer term, development of a new generation of BSCs that can catalytically degrade OPs is needed, while a cost-effective and sustainable alternative source of BSCs must be identified to establish and maintain a strategic reserve. At present, purification of BChE from outdated blood-banked human plasma enables research on how bioscavenger therapy can be used. This stop-gap measure cannot be practically implemented to allow for a sustained supply of that enzyme. It will be necessary to identify a reliable, safe, non supply-limited and inexpensive source of such enzyme.

Stoichiometric or catalytic human enzymes to be used as bioscavengers have to be produced in a eukaryotic system. This is demonstrated by the difficulties of producing human PONs in Escherichia coli (Aharoni et al., 2004) which may lead to unfortunate artifacts (see Corrigendum for (Harel et al., 2004)). Of the candidate bioscavengers, human BChE is the most explored. Several strategies for production of BChE have recently been evaluated, including purification from outdated blood-banked human plasma (Doctor and Saxena, 2005; Grunwald et al., 1997; Lenz et al., 2005) and milk of transgenic goats (Cerasoli et al., 2005). BChE purification from serum (Grunwald et al., 1997) is supply-limited, extremely costly and carries the risk of human-pathogen contamination in the final product. Similarly, production of recombinant cholinesterases in mammalian cell cultures (Velan et al., 1991; Kronman et al., 1992) is also confronted with limited scalability, high costs and risk of pathogen contamination. Recent reports (Cerasoli et al., 2005) describe the use of transgenic goats expressing human BChE in their milk. In a review published by the lead author of that project, the limitations of this technique are clearly delineated and include low efficiency, high cost and lack of a regulatory framework for the production of pharmaceuticals in lower mammalian species (Baldassarre et al., 2004). Additionally, transgenic animals must be consistently maintained at very high numbers because of the long time needed to generate offspring. This implies that production and purification must occur continuously, further contributing to the high cost mentioned above. In addition, mammalian-based production systems seem less promising for large-scale production of AChE-R in particular because of the natural low levels and relative instability of the protein and its cognate mRNA in such systems (Chan et al., 1998; Cohen et al., 2003).

Despite the promise of ChE bioscavengers as effective treatment against NA poisoning, major hurdles exist in making them. The research herein is focused on development of a novel means to biomanufacture ChEs in green plants, which offer a highly scalable and cost-effective production platform.

The inventors have previously expressed and purified acetylcholine esterase (AChE) from plants to obtain an enzyme that is functionally equivalent to the human protein (Mor et al., 2001; Mor and Soreq, 2004; Fletcher et al., 2004; Geyer et al., 2005). However, this system requires months to obtain a sufficient amount and requires the investment of large amounts of land.

### SUMMARY OF THE INVENTION

In some aspects, the present invention relates to compositions and methods for the production of human cholinesterases. More particularly, it relates to methods for the production of human cholinesterases using transient expression and vectors for producing the same.

In one aspect, the present invention relates to a plant viral vector encoding a plant codon-optimized DNA sequence that results in accumulation of the cholinesterase in a plant leaf at levels greater than 20 mg of the enzyme per kilogram of the plant leaf. In some embodiments, the present invention relates to a plant viral vector encoding a plant codon-optimized DNA sequence that results in accumulation of the cholinesterase in a plant leaf at levels less than or equal to 500 mg of the enzyme per kilogram of the plant leaf. The accumulation may be up to or greater than 20, 25, 30, 35, 40, 45, 50, 55, 60, 65, 70, 75, 80, 85, 90, 95, 100, 110, 120, 130, 140, 150, 160, 170, 180, 190, 200, or more mg of the enzyme per kilogram of the plant leaf. In some embodiments, the accumulation may be between 200 mg and 500 mg of the enzyme per kilogram of the plant leaf.

The viral vector may be derived from any suitable source. In particular embodiments, the viral vector is derived from a tobacco mosaic virus. In particular embodiments, the viral vector is derived from a tobamovirus. In other embodiments, the viral vector is derived from a geminivirus, such as a Bean Yellow Dwarf geminivirus.

In some embodiments, the viral vector may contain a signal peptide. In particular embodiments, the signal peptide is a plant signal peptide. For example, the plant signal sequence may be an apoplastic signal peptide, a cytoplasmic signal peptide, or an endogenous signal peptide. In some embodiments, the signal peptide may be a non-native signal peptide. In particular embodiments, the signal peptide may be a non-native mammalian signal peptide. In particular embodiments, the signal peptide may be a synthetic signal sequence. In some embodiments, the synthetic signal peptide controls plant cell localization of the cholinesterase.

The plant may be any suitable plant and may refer generally to a whole plant, or any portion of a plant, including cells, tissues, tissue cultures, seeds, roots, leaves, pollen, and other plant structural components. Numerous types of plants, including both monocotyledonous and dicotyledonous plants, may be modified or engineered within the scope of the plants and method described herein. Non-limiting examples of families of plants that may be used include Solanaceae, Fabaceae (Leguminosae), Chenopodiacae, Brassicaceae, and Graminea. Specific genre of plants that may be used include, but are not limited to, Arabadopsis sp., Brassica sp., Nicotiana sp., Lycopersicon sp., Solanum sp., Medicago sp., Glycine sp., Chenopodium sp., and Spinacia sp., Zea sp., Oryza sp., Hordeum sp. However, it is recognized that these are given as non-limiting examples only. In some embodiments, the plant is Nicotiana benthamiana.

The cholinesterase may be acetylcholinesterase, butylcholinesterase, or variants thereof. In some embodiments, the plant codon-optimized DNA sequence encodes a human acetylcholinesterase. In some embodiments, the plant codon-optimized DNA sequence encodes a human butyrylcholinesterase. In some embodiments, the viral vector is pTM554, pTM697, pTM720, or pTM 840. In some embodiments, disclosed is a viral vector containing a plant codon-optimized DNA sequence that encodes acetylcholinesterase. In some embodiments, disclosed is a viral vector containing a plant codon-optimized DNA sequence that encodes butyrylcholinesterase. In some embodiments, the acetylcholinesterase or the butyrylcholinesterase is a human acetylcholinesterase or butyrylcholinesterase. In some embodiments, the DNA sequence encodes an amino acid sequence has, has at least, or has at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9 percent identity (or any range derivable therein) to a human acetylcholinesterase. In some embodiments, the DNA sequence encodes an amino acid sequence has, has at least, or has at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9 percent identity (or any range derivable therein) to a human butyrylcholinesterase. In some embodiments, the DNA sequence has, has at least, or has at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9 percent identity (or any range derivable therein) to SEQ ID NOS: 1, 3, 5, 7, 9, 11, or 13. In some embodiments, the DNA sequence encodes an amino acid sequence has, has at least, or has at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9 percent identity (or any range derivable therein) to SEQ ID NOS: 2, 4, 6, 8, 10, 12, or 14. In some embodiments, the viral vector contains the nucleic acid sequence of SEQ ID NOS: 1, 3, 5, 7, 9, 11, or 13. In some embodiments, the viral vector contains a nucleic acid sequence which encodes SEQ ID NOS: 2, 4, 6, 8, 10, 12, or 14.

The percent identity between the two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm in sequence-analysis software. Protein analysis software matches similar sequences using measures of similarity assigned to various substitutions, deletions and other modifications, including conservative amino acid substitutions.

In another aspect, the invention relates to a method of transiently producing a cholinesterase using any of the vectors disclosed herein. In some aspects, the invention relates to the use of BChE and its derivatives in hydrolysis of cocaine and succinylcholine (a paralytic).

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

The term “therapeutically effective” as used herein refers to an amount of cells and/or therapeutic composition (such as a therapeutic polynucleotide and/or therapeutic polypeptide) that is employed in methods of the present invention to achieve a therapeutic effect, such as wherein at least one symptom of a condition being treated is at least ameliorated.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is “EPEPE” Episomal Plasmid-Enhanced Protein Expression-Transient expression system based on deconstructed BeYDV (bean yellow dwarf virus).

FIGS. 2A and 2B (a) Schematic model of the EPEPE system. An LSL vector that contains a gene of interest (YFG) driven by a promoter (P) and flanked by a terminator (T) sequence of choice is aroinfiltrated together with a Rep-supplying vector. The LSL vector serves as a “master copy”, which is replicated through a rolling-circle mechanism upon the binding of Rep to the hair-pin structured LIR. Rep is provided in trans from a separate, potentially inducible expression unit to produce high levels of mRNA and translation products. (b) Both Rep and the alternative-splicing product RepA are required for maximal enhancement of expression. An LSL vector containing the gene encoding the Norwalk virus capsid protein was a groin filitrated inton N. benthamiana leaves together with or without (as indicated) a Rep supplying construct (producing both Rep and RepA), an intron-less Rep supplying construct (producing only Rep) or a construct producing p19, PTGS suppressing gene from tomato bushy stunt virus. Maximal expression levels were obtained when both Rep and RepA were present as well as p19. Expression level peaked on day 5 at an outstanding 8% TSP.

FIG. 3 is a graphic summary of the MagnICON system.

FIGS. 4A and B FIG. 4A illustrates the pTM554 vector, which contains a plant optimized BChE gene with its native signal peptide+ER retention signal. FIG. 4B The entire recombinant TMV genome reconstructed following recombination of the 5′ and 3′ modules, nuclear transcription, capping, intron removal, exon splicing and polyadenylation. Cap and poly A tail are not shown. The reconstructed genome contains the following open reading frames: RdRp, encoding two subunits of the RNA-dependent RNA polymerase (wavy light and dark brown lines); MP, encoding the movement protein (wavy orange line); plant optimized gene encoding human BChE with its native signal peptides and an ER retention signal (wavy red line), Note that there are 3 in-frame start codons yielding, potentially, a short (yellow), medium (yellow and blue) and long signal peptides (yellow, blue and purple). Note that there are 3 in-frame start codons yielding, potentially, a short (yellow), medium (yellow and blue) and long signal peptides (yellow, blue and purple).

FIG. 5 illustrates the pTM697 vector.

FIG. 6 illustrates the effects of the 42 amino acid extension on expression. A 3.5-fold increase was seen with the apoplastic module (˜0.016% TSP), and a 7-fold increase was seen with the cytoplasmic module (˜0.032% TSP).

FIG. 7 illustrates pTM720, which contains plant optimized BChE gene with barley α-amylase+ER retention signal. FIG. 7B The entire recombinant TMV genome reconstructed following recombination of the 5′ and 3′ modules, nuclear transcription, capping, intron removal, exon splicing and polyadenylation. Cap and poly A tail are not shown here. The reconstructed genome contains the following open reading frames: RdRp which encodes two subunits of the RNA-dependent RNA polymerase (wavy light and dark brown lines); MP, encoding the movement protein (wavy orange line); plant optimized gene encoding human BChE supplied with α-amylase signal peptide and an ER retention signal (wavy red line). Note that the signal peptide is supplied through recombination with the 5′ module (not shown), followed by transcription of the entire recombinant TMV genome, intron removal and exon splicing.

FIG. 8 illustrates the effect of the endogenous signal peptide compared to a plant-specific signal peptide. The graph shows expression using endogenous or ICON apoplastic signal peptides without the 42 amino acid extension. A 200-fold increase was seen with the ICON apoplastic module (˜6.6% TSP).

FIG. 9 illustrates the effect of the no signal peptide. Removing all signal peptides and the 42 amino acid extension leads to nearly no accumulation. A 2.5-fold decrease was seen when compared to the original construct.

FIGS. 10A-B FIG. 10A illustrates pTM734, which contains plant optimized BChE gene variant (Y332S) with barley α-amylase+6×His His-Tag. FIG. 10B The entire recombinant TMV genome reconstructed following recombination of the 5′ and 3′ modules, nuclear transcription, capping, intron removal, exon splicing and polyadenylation. Cap and poly A tail are not shown here. The reconstructed genome contains the following open reading frames: RdRp which encodes two subunits of the RNA-dependent RNA polymerase (wavy light and dark brown lines); MP, encoding the movement protein (wavy orange line); plant optimized gene encoding an oxime activatable variant of human BChE (wavy red line) supplied with α-amylase signal peptide (yellow) and a 6 His-residue His Tag (6×His, cyan). Site-directed mutation (Y332G) is indicated with a star. Note that the signal peptide is supplied through recombination with the 5′ module (not shown), followed by transcription of the entire recombinant TMV genome, intron removal and exon splicing.

FIGS. 11A-B FIG. 11A illustrates pTM764, which contains plant optimized BChE gene with barley α-amylase+6×His His-Tag. FIG. 11B The entire recombinant TMV genome reconstructed following recombination of the 5′ and 3′ modules, nuclear transcription, capping, intron removal, exon splicing and polyadenylation. Cap and poly A tail are not shown here. The reconstructed genome contains the following open reading frames: RdRp which encodes two subunits of the RNA-dependent RNA polymerase (wavy light and dark brown lines); MP, encoding the movement protein (wavy orange line); plant optimized gene encoding human BChE (wavy red line) supplied with α-amylase signal peptide (yellow) and a 6 His-residue His Tag (6×His, cyan). Note that the signal peptide is supplied through recombination with the 5′ module (not shown), followed by transcription of the entire recombinant TMV genome, intron removal and exon splicing.

FIG. 12 illustrates pTM580, which contains plant optimized BChE gene with endogenous signal peptide+ER retention signal.

FIG. 13 illustrates pTM771, which contains WT BChE in Gemini vector. The plasmid pTM771 is a binary vector (for details describing the pGPTV-Kan backbone) incorporating a T-DNA flanked by left-border (LB) and right-border (RB) sequences and a BeYDV-based replicon directing the expression of a plant-optimized BChE gene. The replicon consists of duplicated Long Intergenic Regions (LIR), a Short Intergenic Region (SIR), C1/C2 open reading that encode through alternative splicing of a short intron (pink) the two BeYDV replication-associated proteins RepA and Rep (Mor et al., 2003). The BChE expression cassette contains the 35S promoter of cauliflower mosaic virus with duplicated enhancer (p35S), the 5′-UTR of tobacco etch virus (TEV), the coding region of mature BChE fused to the signal peptide of tobacco auxin binding protein 1 (ABP1 SP) on its N-terminus and a His-tag on its C-terminus (HIS Tag), followed by the 3′-UTR of the tobacco extension gene (EXT 3′-UTR). with its native signal peptide+ER retention signal. For details describing the pGPTV-Kan backbone see references in (Geyer et al., 2007).

FIG. 14 illustrates pTM775, which is a BeYDV-based vector.

FIGS. 15A-C Transient plant expression of cocaine-hydrolase variants of BChE. Agrobacterium tumefaciens cells harboring the deconstructed TMV-vectors containing the recombinant BChE variant genes (FIG. 15A) were infiltrated by applying vacuum to whole-submerged N. benthamiana plants (FIG. 15B1) or by leaf injection with needle-less syringe into leaves (FIG. 15B2). Plants were harvested at 14-17 days post-infiltration when peak expression is reached (FIG. 15C).

FIGS. 16A-D Variants of BChE designed for cocaine hydrolysis accumulate over time in plants infiltrated with TMV-vectors. Multiple (2 or 3) different leaf samples (0.2 g fresh weight) from different plants were harvested at the given time points. Protein levels determined from the 0.2 g-leaf sample were then extrapolated to determine estimated protein accumulation in 1 kilogram (kg) of fresh leaf material. Mean protein level values±SEM were determined based on activity assays in conjunction with immunoassays from plants infiltrated with MagnICON vectors expressing Variant 2 (FIG. 16A), Variant 3 (FIG. 16B), Variant 4 (FIG. 16C) and Variant 5 (FIG. 16D).

FIGS. 17A-B ConA purified preparations of Variant 4 resolved by SDS-PAGE and subject to Coomassie Staining (FIG. 17A) or Western Blot (FIG. 17B). Lanes (+) and (−) represent a positive control of plant-derived WT BChE and negative control of WT Nicotiana benthamiana. Crude extracts were loaded based on equivalent amounts of total soluble protein. The recombinant protein was partially purified from the initial extract (IE) by 40%-70% ammonium sulfate fractionation (ASF). The protein was then subject to affinity chromatography using Con A-sepharose, eluting with increasing concentrations of methyl-α-D-gluco-pyranoside (E1-E3). E3 corresponds to an 82 fold increase in purity based on specific activity. All Variant 4 pBChE samples were loaded based on equal BChE activity at 240 mU (FIG. 17A) or 2.4 mU (FIG. 17B).

FIG. 18 Plant-derived BChE undergoes substrate activation by its succinylcholine substrate. Blue line represents was fitted by nonlinear regression to fit the equation

$${v}_{0} = ( \frac{1 + b [ SC ] / {K}_{SS}}{1 + [ SC ] / {K}_{SS}} ) ( \frac{{V}_{max}}{1 + {K}_{M} / [ SC ]} )$$

with the following parameters (±SEM): Vₘₐₓ=2.45±0.16 μM/min, K_{M}=57±7 μM, Kₛₛ2.0±0.3 mM, and b=2.9±0.1. K_{cat}=Vₘₐₓ/[BChE]_{T} was calculated to be 516±33 min⁻¹ based on the above Vₘₐₓ value and [BChE]=4.74 nM.

FIGS. 19A-B Plant-derived BChE protects mice from SC-induced apnea. FIG. 19A Respiration rate of mice treated with SC followed by administration of pBChE or saline was monitored. FIG. 19B Survival curve of mice reflecting survival of all pBChE-treated mice as opposed to 100% mortality among control, saline-treated subjects.

FIGS. 20A-C Plant-derived BChE promotes return of normal vital signs in guinea pigs treated with sublethal doses of SC. FIG. 20A Oxygen saturation. FIG. 20B Heart rate. FIG. 20C Time to return to normal heart rate. *, **, and *** denote (respectively) statistically significant, highly significant and extremely significant differences between pBChE-treated and control animals.

FIGS. 21A-C Plant-derived BChE fully protects guinea pigs from high-dose SC-induced apnea. FIG. 21A Oxygen saturation. FIG. 21B Heart rate. FIG. 21C Time to return to normal heart rate. At time points beyond 3 minutes, there existed extremely significant differences between pBChE-treated and control animals.

FIG. 22 illustrates pTM 840-WT BChE-50 amino acid from C-Terminal.

### DETAILED DESCRIPTION OF ILLUSTRATIVE EMBODIMENTS

The methods and compositions disclosed herein utilize tobacco plants as a production system for research quantities of human cholinesterases (ChE) that have strong potential as bioscavengers (BSC) against organophsphate (OP) chemical warfare nerve agents.

### A. PLANT EXPRESSION SYSTEMS FOR PROTEIN PHARMACEUTICALS

Currently, there are two major methods to express foreign antigens in plants: transient expression and genetic transformation.

Plant viruses in general are very efficient pathogens, employing very compact genomes to subject their host cells to make large quantities of a limited set of proteins. Consequently, viral expression vectors were devised to allow efficient transient expression of recombinant proteins in plants (Timmermans et al., 1994; Scholthof et al., 1996; Palmer and Rybicki, 1997). These vectors are in essence transgenic viruses, which are used for infection of plants and express a recombinant product as they spread throughout the plant. Among the various vectors created for this purpose those based on tobamoviruses (e.g. tobacco mosaic virus) have received most attention (Turpen, 1999) and have been useful in directing expression of foreign genes (with some size limitations) (McCormick et al., 1999) or as coat-protein peptide fusions (Yusibov et al., 1997). Other plant viruses can also be utilized and vectors have been created based on e.g. plum pox virus (Fernandez-Fernandez et al., 2001), cowpea mosaic virus (Brennan et al., 1999; Brennan et al., 2001; Dalsgaard et al., 1997; Durrani et al., 1998; Gilleland et al., 2000; McInerney et al., 1999), alfalfa mosaic virus (Yusibov et al., 1997; Belanger et al., 2000), cucumber mosaic virus (Natilla et al., 2004), tomato bushy stunt virus (Joelson et al., 1997) and potato virus X (Brennan et al., 1999; Franconi et al., 2002; Marusic et al., 2001; O'Brien et al., 2000). In most cases, by using viral vectors researchers could achieve increased accumulation of the recombinant antigens, sometimes 1000-fold more. However, this expression system does have its drawbacks including genetic instability (the viral vectors often recombine to exclude the inserted foreign sequences and regenerate the WT virus), the need to individually inoculate each plant, and environmental concerns regarding the release of agronomically important plant pathogens.

As alternative, several groups have been developing “deconstructed” viral systems (Gleba et al., 2004), such that make use of certain essential elements of the plant virus but dispense with undesirably limiting elements, thus offering improved performance and safety. Here the inventors will explore two such transient expression systems (Mor et al., 2003; Zhang et al., 2006; Santi et al., 2006; Marillonnet et al., 2004; Gleba et al., 2005).

1. Episomal Plasmid-Enhanced Protein Expression

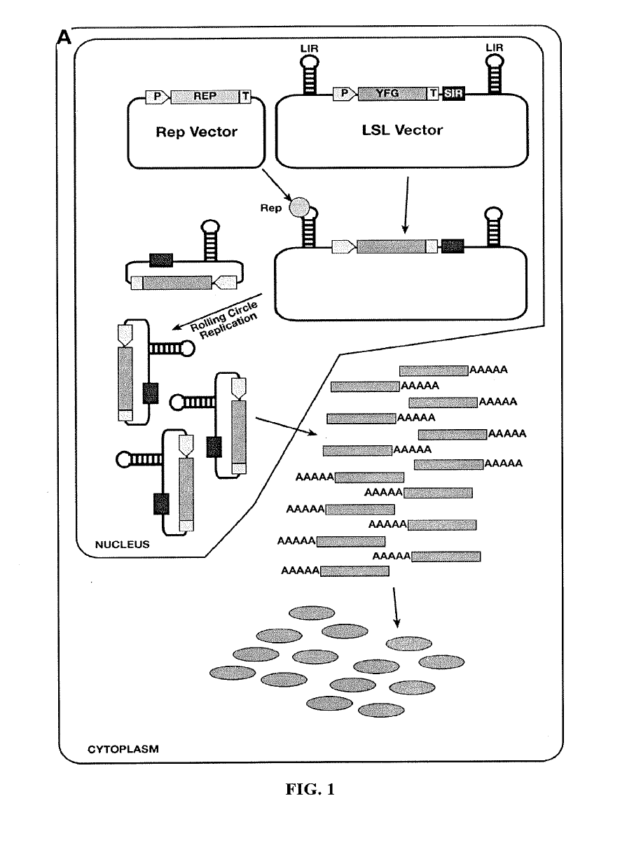

The inventors have recently developed a gene amplification system for enhancement of foreign protein expression in plants called Episomal Plasmid-Enhanced Protein Expression (EPEPE) (FIG. 1). It is based on the replication machinery of a monopartite geminivirus, Bean Yellow Dwarf Virus (BeYDV) (Mor et al., 2003). BeYDV is a geminivirus of the Mastrevirus subgroup with a single genome component of single stranded circular DNA that replicates to very high copy number in the nuclei of infected cells (Liu et al., 1998). The general strategy is to utilize two vectors comprising different portions of the BeYDV genome that interact in their function to achieve enhanced protein expression. The system can either be used for transient expression without integration into the plant chromosome (Mor et al., 2003) or incorporated in the plant genome to generate a transgenic plant or tissue (Zhang et al., 2006). In either case, the desired protein expression occurs as a result of the accumulation of multi-copy episomal plasmids encoding the gene for the desired protein.

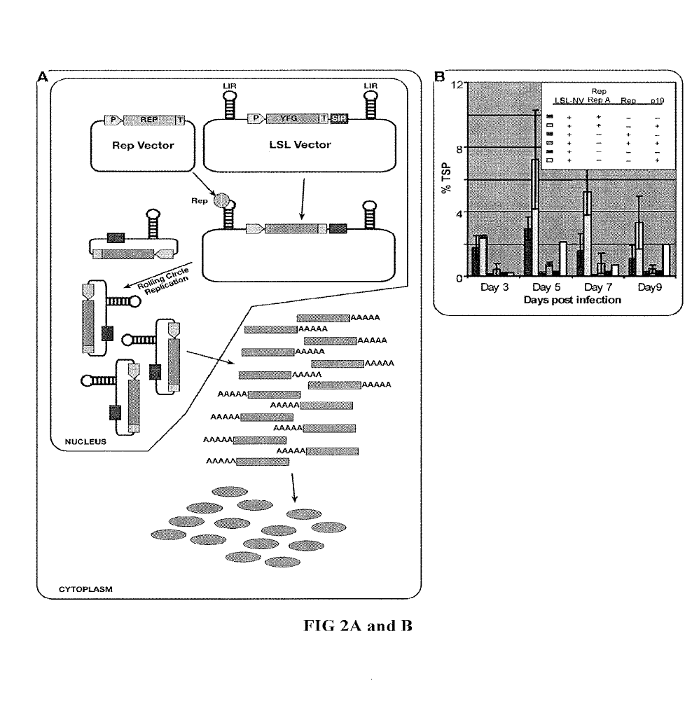

The BeYDV replication initiator protein, Rep and the alternative-splicing variant RepA are essential for replication but act in-trans; therefore Rep/RepA can be supplied from another plasmid (the “Rep supplying vector”) or from a nuclear transgene. This is the first of the two EPEPE vectors. The second vector, the “LSL replicon” contains the minimal cis-acting elements required for replication: the short (SIR) and long (LIR) intergenic regions (FIG. 2). The LSL replicon cassette is flanked on either side by the LIR, with the SIR lying in between. An expression cassette with a gene of interest is inserted on one side of the SIR (e.g. the CaMV 35S promoter driving the gene encoding the capsid protein of Norwalk virus (NV) in FIG. 2). Using argroinfiltration of both the LSL-NV and a Rep/RepA supplying construct into N. benthamiana leaves, a 10-20 fold increase of expression of the NV coat protein (assayed by ELISA) as compared to the expression in the LSL-NV only control. Interestingly, both Rep and RepA seem to be required to obtain maximal expression levels. Similar results were previously reported by us for transient expression in tobacco “NT1” cell cultures (Mor et al., 2003).

2. MagnICON

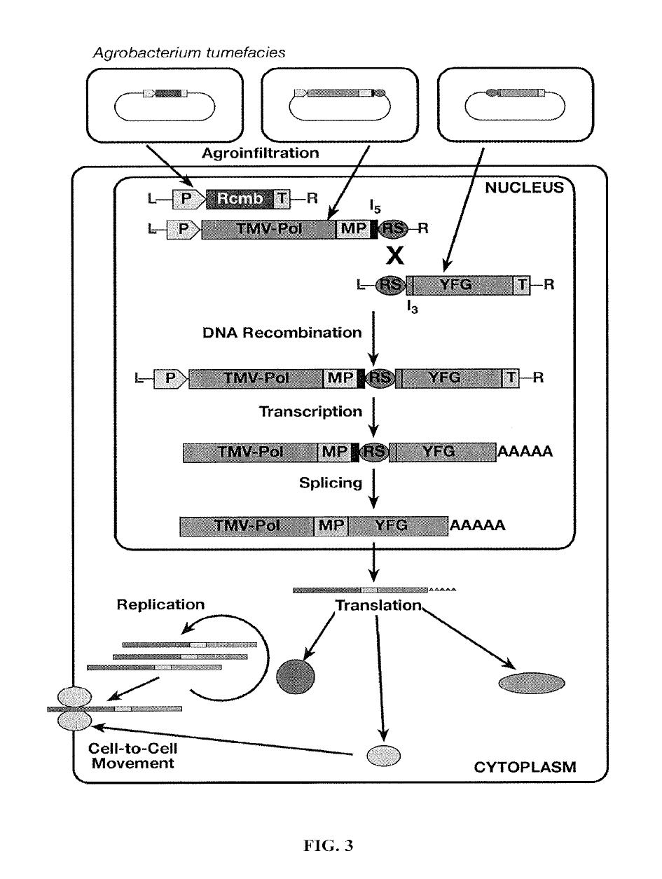

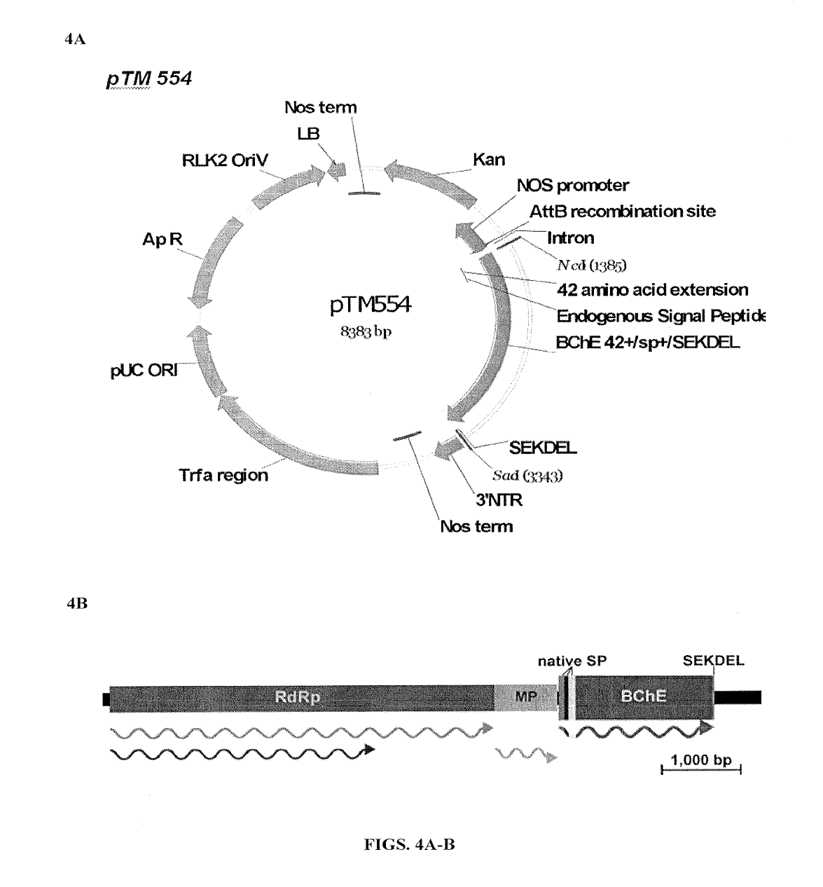

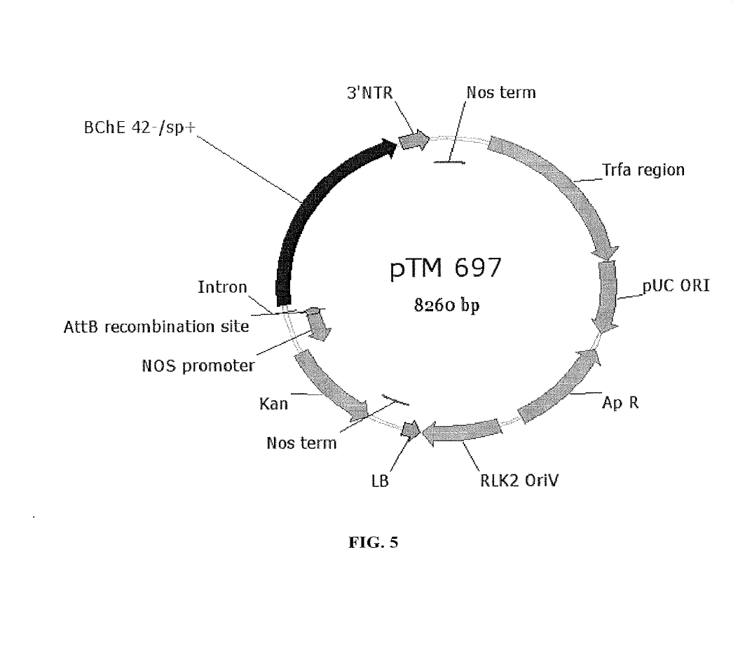

The MagnICON® system (ICON Genetics, Princeton, N.J.), is based on the well-studied TMV virus. Like other tobamoviruses, TMV is a (+)RNA virus, which completes its replicative cycle in the cytoplasm of the infected plant cell. Infection therefore necessitated complex cloning into whole virus genome, in vitro transcription and inefficient mechanical inoculation of naked RNA. In contrast, the use of Agrobacterium tumefaciens to deliver DNA vectors into plant cells (i.e. “agroinfect”) is a very efficient process but one that required re-engineering parts of the TMV genome to allow it first to be effectively transcribed in the nucleus, correctly processed and enter the cytoplasm to be translated. From there the virus, subsequent to many rounds of cytoplasmic replication, can move and infect neighboring cells. (FIG. 3)

Magnfection has a deletion of the TMV coat protein, thus limiting its ability to spread systemically throughout the plant but maintaining the ability of the virus move cell-to-cell. This innovation at once allows larger recombinant genes to be expressed (encapsidation limits genome size) and establishes a stringent containment of the virus. The host plant system the inventors use is N. benthamiana, a non-food and non-feed crop (S anti et al., 2006; Marillonnet et al., 2005; Gleba et al., 2005). The system yields outstanding levels of foreign protein in the range of 1-5 mg per gram of plant material (Santi et al., 2006; Marillonnet et al., 2005; Gleba et al., 2005).

### B. CHOLINESTERASES

### 1. AChE

For almost as many years as acetylcholine (ACh) has been recognized as a neurotransmitter, the vital role of the acetylcholine-hydrolyzing enzyme, acetylcholinesterase (AChE), in terminating cholinergic neurotransmission has been recognized. Research of AChE is intimately linked to the study of its inhibitors. The realization of the vulnerability of this enzyme promoted the discovery and synthesis of inhibitors for use as pesticides, therapeutics and, unfortunately, as chemical-warfare agents. These inhibitors have helped to elucidate AChE's function and mode of action and this knowledge, in turn, was used in the design of even more potent inhibitors.

The best known function of AChE is the termination of neurotransmission in cholinergic synapses controlling skeletal muscles, autonomic functions (e.g., heartbeat, exocrine glands, and smooth muscles) and many central pathways in the brain. To ensure a discrete “all-or-none”response across the synapse, the release of acetylcholine is tightly controlled and the neurotransmitter is efficiently hydrolyzed by AChE (Taylor and Radic, 1994; Schwarz et al., 1995; Massoulie et al., 1999; Grisaru et al., 1999; Soreq and Seidman, 2001). The catalytic mechanism of AChE and its critical role make the enzyme vulnerable to a variety of inhibitors. While some naturally occurring AChE inhibitors are very potent, human exposure to them is rare. However, manmade anti-AChE compounds, especially OPs are widely used as pesticides and pose a substantial occupational and environmental risk (Marrs, 1993; Sultatos, 1994; Millard and Broomfield, 1995). Even more ominous is the fear of deliberate use of OPs as chemical warfare agents against individuals or populations by terrorists or by governments that defy international conventions (Gunderson et al., 1992; Nagao et al., 1997).

Inhibition of synaptic acetylcholinesterase (AChE-S) leads to the accumulation of ACh in the synapse and causing neural over-stimulation (Soreq and Seidman, 2001). The severity of the ensuing nicotinic and muscarinic symptoms is dose-dependent and can result in death due to cardiovascular and respiratory collapse (Greenfield et al., 2002; Lee, 2003). Those surviving the initial insult often suffer long-term sequelae, including OP-induced delayed neuropathy, muscle weakness, permanent brain dismorphology and social/behavioral deficits (Greenfield et al., 2002; Lee, 2003; Yamasue et al., 2003). The mechanisms underlying OP-delayed toxicity and other stressful insults, involve rapid elevation of c-fos followed by upregulated expression the ACHE gene (Friedman et al., 1996) and rapid, yet long-lasting shifted alternative splicing from AChE-S to the otherwise rare “readthrough” variant (AChE-R) (Kaufer et al., 1998). Expression of AChE-R also continues for weeks following psychologically stressful events or head trauma (Shohami et al., 2000; Meshorer et al., 2002). Upregulation and isoform switching are associated with short-term neuroprotection, however, prolonged overexpression of AChE-R exerts long-lasting damage (Soreq and Seidman, 2001). NMJs show degenerated synaptic folds, enlarged motor endplates, disorganized muscle fibers and branded terminal nerves (Lev-Lehman et al., 2000), accompanied by neuromuscular malfunctioning (Farchi et al., 2003). Thus the goal of any successful therapy should the prevention of the immediate life-threatening effects of OP intoxication and its long-term debilitating consequences.

Anticholinesterase OPs are hemisubstrates, that is, they form stable covalent enzyme-OP adducts in a similar way to the covalent (but transient) bond between acetate and the ChE (Taylor, 1996). The active-site serine residue is rapidly phosphylated, but unlike the case of acetate, the enzyme is regenerated only extremely slowly. Dealkylation of the conjugated OP (“aging”) further stabilizes the inhibited enzyme and significant spontaneous dephosphorylation of the serine is not observed. Aging of an alkoxy group renders the enzyme recalcitrant to reactivation.

As discussed in detail above, acetylcholinesterase (EC 3.1.1.7, GenBank Accession No. P22303) is well known in the art. Furthermore, several natural variants are also known, e.g., Isoform T (GenBank Accession No. P22303-1), Isoform H (GenBank Accession No. P22303-2), and Isoform R (GenBank Accession No. P22303-4).

### 2. BChE and CaE

AChE and butyrylcholinesterase (BChE) belong to the α/β fold hydrolase family. These hydrolases bind OP anticholinesterases very efficiently, however the phosphorylated enzyme fails to reactivate. Some mutants of BChE were shown to reactivate much less slowly, effectively making them OP hydrolases (Millard et al., 1995; Millard et al., 1998; Lockridge et al., 1997; Broomfield et al., 1999). Mutations that reduce the rate of the dealkylation aging process in both BChE (Millard et al., 1998) and AChE (Maxwell et al., 1999) were previously identified. Although significant improvements were achieved, these mutated hydrolases were still not satisfactory. Carboxylesterase (CaE) is another member of the same family with broader catalytic activities which is sensitive to OPs. However, CaE can self activate, and it is thought that a naturally occurring H is residue within the sequence WIHGGGL plays a role in the process. The corresponding sequence of BChE (and AChE) is WIYGGGF. One of the OP-hydrolase enhancing mutations, G117H of BChE, is in the same region (WIYGGHF). Unlike PON1 and the ChE enzymes, CaE, is not normally found in human serum (Li et al., 2005). The OP-hydrolyzing activities associated with native CaEs, murine BChE, and recombinant human BChE raises the option of evolving these enzymes into more efficient phosphortriesterases. Butyrylcholinesterase is known in the art (EC 3.1.1.8, GenBank Accession No. P06276).

### C. SIGNAL PEPTIDES

Most secretory and plasma membrane proteins utilize a co-translation/translocation system which relies on well-conserved membrane bound translocons called Sec61p in eukaryotes and secYEG in archaea and bacteria (Saraogi and Shan, 2011 and references therein). Translationally-arrested ribosomes together with their bound substrate mRNA and the nascent protein are targeted to the translocon by a Signal Recognition Particle (SRP) and membrane-associated SRP receptor, which are also well-conserved between prokaryotes and eukaryotes (Saraogi and Shan, 2011). The SRP binds to the N-terminal region of the nascent protein as it is being extruded from the translating ribosome (Imai and Nakai, 2010; Saraogi and Shan, 2011). Thus, the information to target a protein from the cytosol to the plasma membrane of prokaryotes or to the endoplasmic reticulum (ER) membrane of eukaryotes is typically contained in the N-terminal domain, the so-called “signal peptide” of the cargo protein. Signal peptides are usually 15-25 residues long with an obligatory core of 8-12 hydrophobic amino acids (h-region), which is often, but not always, flanked by a positively charged n-region and a polar c-region, where the signal peptide is cleaved off the protein once it passes through the translocon. Aside from these very loosely defining characteristics, signal peptides are quite variable. It is often possible to predict with a reasonable accuracy that a certain amino acid sequence can serve as a signal peptide and several prediction algorithms were developed for that purpose with SignalP (available on the world wide web at cbs.dtu.dk/services/SignalP) being the most accurate (Bendtsen et al., 2004; Choo et al., 2009). It is difficult, however, given the current state of knowledge to predict the relative efficiencies of the signal peptides.

A practical advantage of this broad-stroke sequence conservation of signal peptides is that a signal peptide from one organism operates well in the context of heterologous expression transgenic host. Thus for example the inventors have had success in expressing human acetylcholinesterase (AChE) and butyrylcholinesterase (BChE) in transgenic plants where their native (human) signal peptide directed their accumulation in the ER to about 1% of total soluble protein (TSP, Evron et al., 2007; Geyer et al., 2007; Geyer et al., 2010). However, as stated above, the efficiency by which different signal peptides drives the targeting to their cargo proteins into the ER, or the targeting efficiency of a certain signal peptide in different expression hosts or under different conditions, have to be determined empirically at this stage. For example the use of the native human signal peptide of BChE that performed very well in the transgenic context was much less effective when transiently expressed using a tobacco mosaic virus expression system in the same plant species (Nicotiana benthamiana) achieving a level of only 0.032% TSP (more than 30 fold lower levels). Much higher levels of >6% were achieved when a plant signal peptide (from the gene encoding barley alpha amylase) was used instead. These levels are 200 fold higher than those obtained by using the native signal peptide for transient expression and 6 fold higher than those obtained with transgenic plants. In fact under such conditions the protein accumulates to about 232 mg BChE per 1 kg (fresh weight) leaf material.

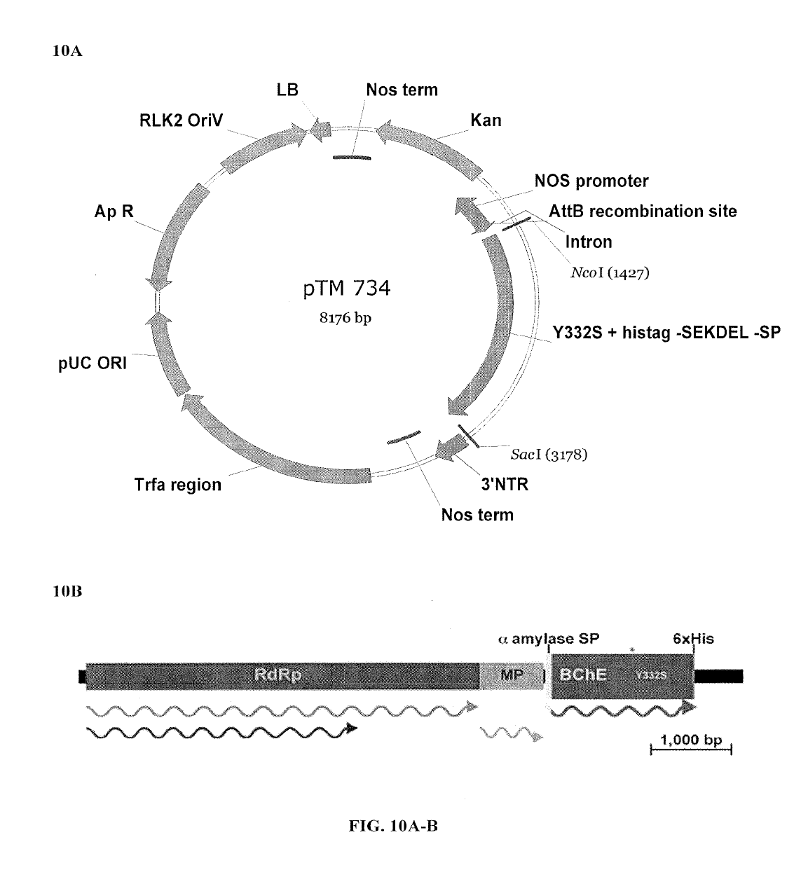

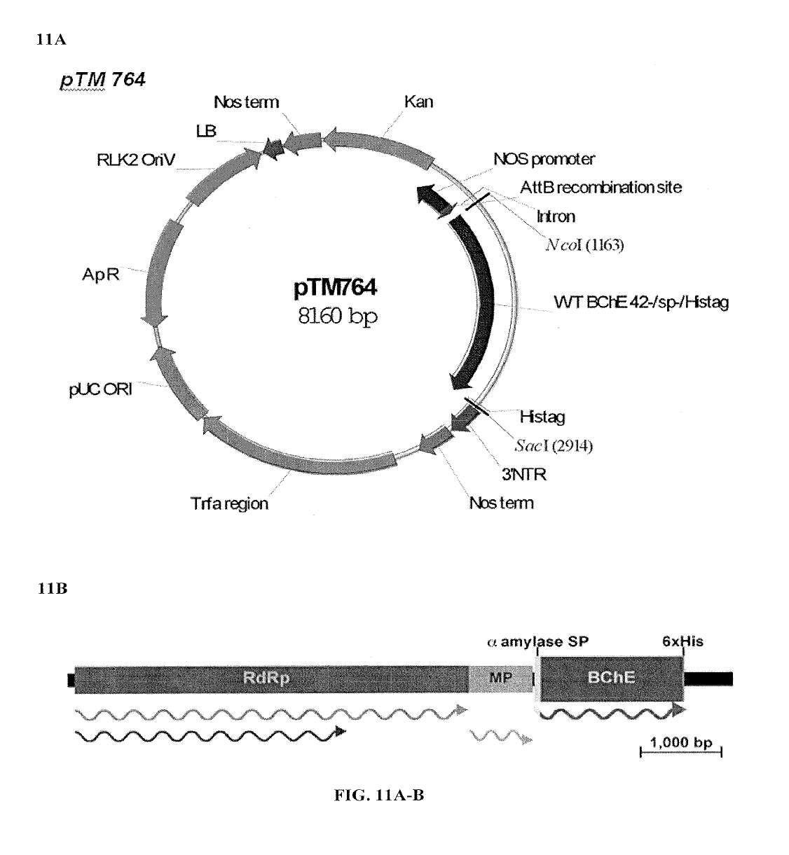

Following entry into the ER, a protein can be targeted to many different compartments inside the endo-membrane system (ER and Golgi), directed to other organelles (e.g., vacuoles and peroxisomes), intergrated into the plasma membrane or secreted to the apoplastic space. In the absence of additional targeting information (beyond the SP), integral membrane proteins will reach the plasma membrane and soluble proteins will be secreted. Proteins that carry an ER-retention signal consisting of the short peptide KDEL (or sometimes HDEL and SEKDEL) on their C-termini, will be captured by KDEL-receptor resident in the cis-Golgi and will be recycled back into the ER, thus prevented from continuing their journey down the secretory pathway. Experience demonstrated that ER-retention often increases the accumulation of a recombinant protein produced in a heterologous transgenic host (Evron et al., 2007; Geyer et al., 2007; Geyer et al., 2010). In the context of virus-assisted transient expression it was assumed that similar requirements prevail, however BChE, directed to the ER, but is devoid of an ER retention signal. This requirement seems to be less rigorous as the inventors show here for a variant of BChE (Y332S, pTM734 (FIG. 10)) that accumulates to 3.5% TSP, whereas its counterpart that has SEKDEL peptide on its C-terminus (but otherwise identical), accumulated substantially less than 1% TSP.

### D. NUCLEIC ACID-BASED EXPRESSION SYSTEMS

Nucleic acid-based expression systems may find use, in certain embodiments of the invention.

1. Methods of Nucleic Acid Delivery

Suitable methods for nucleic acid delivery for transformation of a cell are believed to include virtually any method by which a nucleic acid (e.g., DNA) can be introduced into such a cell, or even an organelle thereof. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); or by agitation with silicon carbide fibers (Kaeppler et al., 1990; U.S. Pat. Nos. 5,302,523 and 5,464,765, each incorporated herein by reference); by desiccation/inhibition-mediated DNA uptake (Potrykus et al., 1985). Through the application of techniques such as these, cells may be stably or transiently transformed.

a. Electroporation

In certain embodiments of the present invention, a nucleic acid is introduced into a cell via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

b. Calcium Phosphate

In other embodiments of the present invention, a nucleic acid is introduced to the cells using calcium phosphate precipitation.

2. Vectors

Vectors may find use with the current invention. In one embodiment of the invention, an entire heterogeneous “library” of nucleic acid sequences encoding target polypeptides may be introduced into a population of bacteria, thereby allowing screening of the entire library. The term “vector” is used to refer to a carrier nucleic acid molecule into which a nucleic acid sequence can be inserted for introduction into a cell where it can be replicated. A nucleic acid sequence can be “exogenous,” or “heterologous”, which means that it is foreign to the cell into which the vector is being introduced or that the sequence is homologous to a sequence in the cell but in a position within the host cell nucleic acid in which the sequence is ordinarily not found. Vectors include plasmids, cosmids and viruses (e.g., bacteriophage). One of skill in the art may construct a vector through standard recombinant techniques, which are described in Maniatis et al., 1988 and Ausubel et al., 1994, both of which references are incorporated herein by reference.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference).

Naturally, it will be important to employ a promoter and/or enhancer that effectively directs the expression of the DNA segment in the cell type chosen for expression. One example of such promoter that may be used with the invention is the E. coli arabinose or T7 promoter. Those of skill in the art of molecular biology generally are familiar with the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

c. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Termination Signals

The vectors or constructs prepared in accordance with the present invention will generally comprise at least one termination signal. A “termination signal” or “terminator” is comprised of the DNA sequences involved in specific termination of an RNA transcript by an RNA polymerase. Thus, in certain embodiments, a termination signal that ends the production of an RNA transcript is contemplated. A terminator may be necessary in vivo to achieve desirable message levels.

Terminators contemplated for use in the invention include any known terminator of transcription described herein or known to one of ordinary skill in the art, including but not limited to, for example, rho dependent or rho independent terminators. In certain embodiments, the termination signal may be a lack of transcribable or translatable sequence, such as due to a sequence truncation.

e. Origins of Replication

In order to propagate a vector in a host cell, it may contain one or more origins of replication sites (often termed “ori”), which is a specific nucleic acid sequence at which replication is initiated.

f. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

3. Binary Vector Systems

In a binary vector system, two different plasmids are employed. The first is a wide-host-range small replicon, which has an origin of replication (ori) that permits the maintenance of the plasmid in a wide range of bacteria including Agrobacterium. This plasmid typically contains foreign DNA in place of T-DNA, the left and right T-DNA borders (or at least the right T-border), markers for selection and maintenance in A. tumefaciens, and a selectable marker for plants. The plasmid is said to be “disarmed”, since its tumor-inducing genes located in the T-DNA have been removed. The second is a helper Ti plasmid, harbored in A. tumefaciens, which lacks the entire T-DNA region but contains an intact vir region.

In general, the recombinant small replicon is transferred via bacterial conjugation or direct transfer to A. tumefaciens harboring a helper Ti plasmid, and the plant cells are co-cultivated with the Agrobacterium, to allow transfer of recombinant T-DNA into the plant genome, and transformed plant cells are selected under appropriate conditions.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### TMV-Based Vector Constructs

1. pTM554

The inventors first created pTM554, which contained a plant-optimized BChE gene with its native signal peptide, an ER retention signal, and a 42 amino acid extension (FIG. 4A; SEQ ID NOS:1 and 2). The reconstructed genome contains the following open reading frames: RdRp, encoding two subunits of the RNA-dependent RNA polymerase; MP, encoding the movement protein; plant optimized gene encoding human BChE with its native signal peptides and an ER retention signal, Note that there are 3 in-frame start codons yielding, potentially, a short, medium and long signal peptides. FIG. 4B. In the nucleic acid sequence below (SEQ ID NO:1), bold italics indicates the alternative start codons, bold underline indicates the long endogenous signal peptide, underlined indicates the short endogenous signal peptide, and underlined italics indicates the ER retention signal:

[symbol]

ggatctgtgcaaagcaacctccaagctggagctgctgctgccagctgcatctccccaaagtactac [symbol] at
cttcactccttgcaagctctaccacctctgttgtagggagtctgagatcaac [symbol] cacagcaaggttaccatcattt
gcatcaggttcctcttttggttcctcctcctctgcatgcttattggtaagagccacactgaggatgacatcatcattgccac
caagaatggtaaggttaggggtatgaacctcacagtttttggtggtactgttacagccttccttggtattccttatgcccaa
ccacctcttggtagacttaggttcaagaagccacaaagcctcaccaagtggtctgacatttggaatgccaccaagtatg
ccaactcctgttgtcaaaacattgaccaatccttcccaggatttcatggatctgagatgtggaacccaaacactgacctct
ctgaggattgtctttaccttaatgtgtggatcccagccccaaagcctaagaatgccactgttctcatttggatctatggtgg
tggtttccaaactggaacctcctctctccatgtttatgatggaaagttcttggctagagttgagagagttattgtggtgagc
atgaactatagggtgggtgccttgggattcttggccctcccaggaaatcctgaggccccaggtaatatgggtctttttga
ccaacaattggctcttcaatgggttcagaagaacattgctgcctttggtggaaaccctaagtctgttaccctctttggaga
gtctgctggagctgcttctgttagccttcacttgctttctcctggaagccactccttgttcactagagccattctccaatctg
gatccttcaatgctccttgggctgtgacatctctttatgaggctaggaatagaacattgaaccttgctaagttgactggttg
ctctagagagaatgagactgagatcatcaagtgtcttagaaacaaggacccacaagagattcttttgaatgaggcctttg
ttgttccttatggaacccctttgtctgtgaactttggtcctacagtggatggtgatttcctcactgacatgccagacatcttgc
ttgagcttggacaattcaagaagacccaaattttggtgggtgttaacaaggatgagggtacagctttccttgtgtatggcg
cgcctggttttagcaaggacaacaactccatcatcactagaaaggagttccaagagggtctcaagatcttcttcccagg
agtgtctgagtttggaaaggagtccatccttttccattacacagattgggttgatgaccaaagacctgagaactataggg
aggccttgggtgatgttgttggagattacaacttcatttgccctgccttggagttcaccaagaagttctctgagtggggaa
ataatgccttcttctactactttgagcataggtcctccaagctcccttggccagagtggatgggagtgatgcatggttatg
agattgagtttgtttttggtttgcctcttgagagaagagataactacacaaaggctgaggagatcttgagcagatccattgt
gaagaggtgggccaactttgccaagtatggtaatccaaatgagactcaaaacaatagcacaagctggcctgtgttcaa
gagcactgagcaaaagtacctcaccttgaacacagagtccacaaggattatgaccaagttgagggctcaacaatgtag
gttttggacatccttcttcccaaaggtgttggagatgacaggaaatatcgatgaggctgagtgggagtggaaggctgga
ttccataggtggaacaactacatgatggattggaagaaccaattcaatgattacactagcaagaaggagagctgtgtgg
gtctctctgagaaggatgaactctag

In the peptide sequence below (SEQ ID NO:2), bold italics indicates the alternative initiatory methionine residues, bold underline indicates the long endogenous signal peptide, underlined indicates the short endogenous signal peptide, and underlined italics indicates the ER retention signal.

[symbol]

GSVQSNLQAGAAAASCISPKYY [symbol] IFTPCKLYHLCCRESEIN [symbol]
HSKVTIICIRFLFWFLLLCMLIGKSHTEDDIIIATKNGKVRGMNLTV
FGGTVTAFLGIPYAQPPLGRLRFKKPQSLTKWSDIWNATKYANSCCQNI
DQSFPGFHGSEMWNPNTDLSEDCLYLNVWIPAPKPKNATVLIWIYGGGF
QTGTSSLHVYDGKFLARVERVIVVSMNYRVGALGFLALPGNPEAPGNMG
LFDQQLALQWVQKNIAAFGGNPKSVTLFGESAGAASVSLHLLSPGSHSL
FTRAILQSGSFNAPWAVTSLYEARNRTLNLAKLTGCSRENETEIIKCLR
NKDPQEILLNEAFVVPYGTPLSVNFGPTVDGDFLTDMPDILLELGQFKK
TQILVGVNKDEGTAFLVYGAPGFSKDNNSIITRKEFQEGLKIFFPGVSE
FGKESILFHYTDWVDDQRPENYREALGDVVGDYNFICPALEFTKKFSEW
GNNAFFYYFEHRSSKLPWPEWMGVMHGYEIEFVFGLPLERRDNYTKAEE
SILRSIVKRWANFAKYGNPNETQNNSTSWPVFKSTEQKYLTLNTESTRI
MTKLRAQQCRFWTSFFPKVLEMTGNIDEAEWEWKAGFHRWNNYMMDWKN
QFNDYTSKKESCVGLSEKDEL

The pTM554 vector was initially used for the transient expression of BChE in Nicotiana benthamiana leaf using the MagnICON system (FIG. 4). Considerable necrosis was seen at 7 days post infection (DPI).

2. pTM720

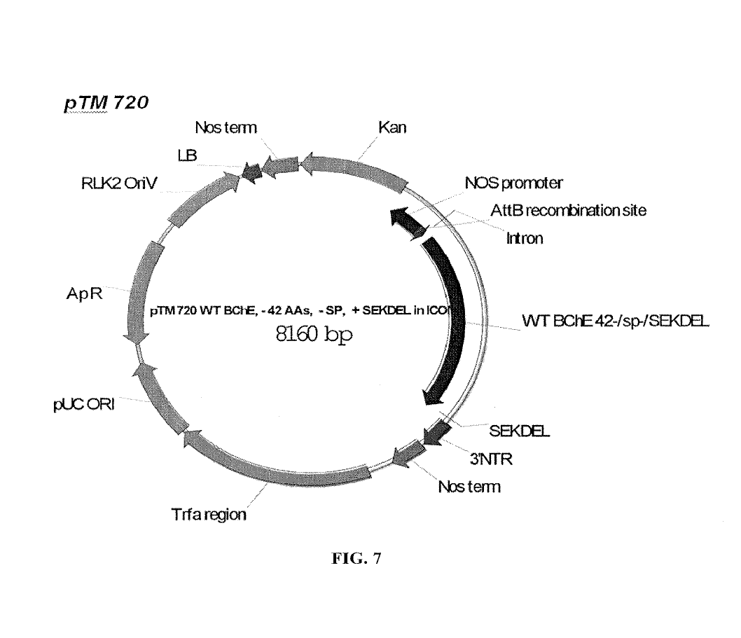

The inventors then removed the 42 amino acid extension and added a plant-specific signal peptide (α-amylase) to create vector pTM720 (FIGS. 7A-B; SEQ ID NOS:3 and 4). The reconstructed genome contains the following open reading frames: RdRp which encodes two subunits of the RNA-dependent RNA polymerase; MP, encoding the movement protein; plant optimized gene encoding human BChE supplied with α-amylase signal peptide and an ER retention signal (SEKDEL). FIG. 7B. In the nucleic acid sequence below (SEQ ID NO:3), bold italics indicates the start codon, bold underline indicates the α-amylase signal peptide, and underlined italics indicates the ER retention signal:

[symbol]

gcgaacaaacacttgtccctctccctcttcctcgtcctccttggcctgtcggccagcttggcctccggagccat
ggaggatgacatcatcattgccaccaagaatggtaaggttaggggtatgaacctcacagtttttggtggtactgttacag
ccttccttggtattccttatgcccaaccacctcttggtagacttaggttcaagaagccacaaagcctcaccaagtggtctg
acatttggaatgccaccaagtatgccaactcctgttgtcaaaacattgaccaatccttcccaggatttcatggatctgagat
gtggaacccaaacactgacctctctgaggattgtctttaccttaatgtgtggatcccagccccaaagcctaagaatgcca
ctgttctcatttggatctatggtggtggtttccaaactggaacctcctctctccatgtttatgatggaaagttcttggctagag
ttgagagagttattgtggtgagcatgaactatagggtgggtgccttgggattcttggccctcccaggaaatcctgaggcc
ccaggtaatatgggtctttttgaccaacaattggctcttcaatgggttcagaagaacattgctgcctttggtggaaacccta
agtctgttaccctctttggagagtctgctggagctgcttctgttagccttcacttgctttctcctggaagccactccttgttca
ctagagccattctccaatctggatccttcaatgctccttgggctgtgacatctctttatgaggctaggaatagaacattgaa
ccttgctaagttgactggttgctctagagagaatgagactgagatcatcaagtgtcttagaaacaaggacccacaagag
attcttttgaatgaggcctttgttgttccttatggaacccctttgtctgtgaactttggtcctacagtggatggtgatttcctca
ctgacatgccagacatcttgcttgagcttggacaattcaagaagacccaaattttggtgggtgttaacaaggatgagggt
acagctttccttgtgtatggcgcgcctggttttagcaaggacaacaactccatcatcactagaaaggagttccaagagg
gtctcaagatcttcttcccaggagtgtctgagtttaggaaaggagtccatcatttccattacacagattgggttgatgacca
aagacctgagaactatagggaggccttgggtgatgttgttggagattacaacttcatttgccctgccttggagttcaccaa
gaagttctctgagtggggaaataatgccttcttctactactttgagcataggtcctccaagctcccttggccagagtggat
gggagtgatgcatggttatgagattgagtttgtttttggtttgcctcttgagagaagagataactacacaaaggctgagga
gatcttgagcagatccattgtgaagaggtgggccaactttgccaagtatggtaatccaaatgagactcaaaacaatagc
acaagctggcctgtgttcaagagcactgagcaaaagtacctcaccttgaacacagagtccacaaggattatgaccaag
ttgagggctcaacaatgtaggttttggacatccttcttcccaaaggtgttggagatgacaggaaatatcgatgaggctga
gtgggagtggaaggctggattccataggtggaacaactacatgatggattggaagaaccaattcaatgattacactagc
aagaaggagagctgtgtgggtctctctgagaaggatgaactctag

In the peptide sequence below (SEQ ID NO:4), bold italics indicates the alternative initiatory methionine residue, bold underline indicates the α-amylase signal peptide, and underlined italics indicates the ER retention signal.

[symbol]

ANKHLSLSLFLVLLGLSASLASGAMEDDIIIATKNGKVRGMNLTVF
GGTVTAFLGIPYAQPPLGRLRFKKPQSLTKWSDIWNATKYANSCCQNID
QSFPGFHGSEMWNPNTDLSEDCLYNVWIPAPKPKNATVLIWIYGGGFQT
GTSSLHVYDGKFLARVERVIVVSMNYRVGALGFLALPGNPEAPGNMGLF
DQQLALQWVQKNIAAFGGNPKSVTLFGESAGAASVSLHLLSPGSHSLFT
RAILQSGSFNAPWAVTSLYEARNRTLNLAKLTGCSRENETEIIKCLRNK
DPQEILLNEAFVVPYGTPLSVNFGPTVDGDFLTDMPDILLELGQFKKTQ
ILVGVNKDEGTAFLVYGAPGFSKDNNSIITRKEFQEGLKIFFPGVSEFG
KESILFHYTDWVDDQRPENYREALGDVVGDYNFICPALEFTKKESEWGN
NAFFYYFEHRSSKLPWPEWMGVMHGYEIEFVFGLPLERRDNYTKAEEIL
SRSIVKRWANFAKYGNPNETQNNSTSWPVFKSTEQKYLTLNTESTRIMT
KLRAQQCRFWTSFFPKVLEMTGNIDEAEWEWKAGFHRWNNYMMDWKNQF
NDYTSKKESCVGLSEKDEL

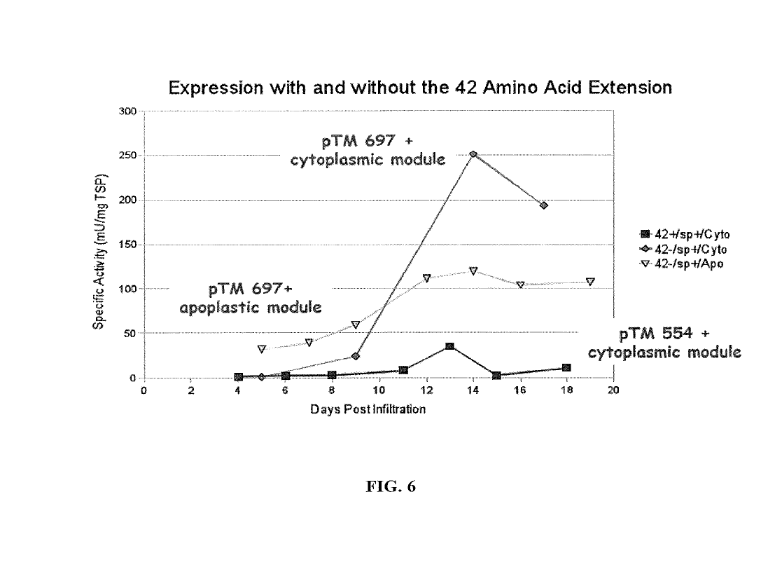

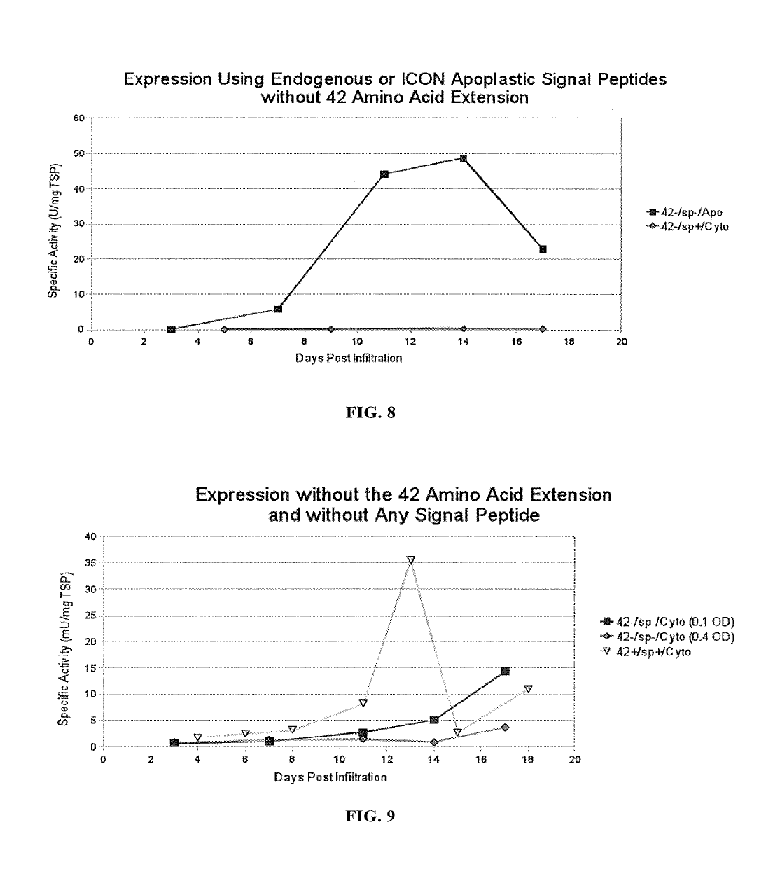

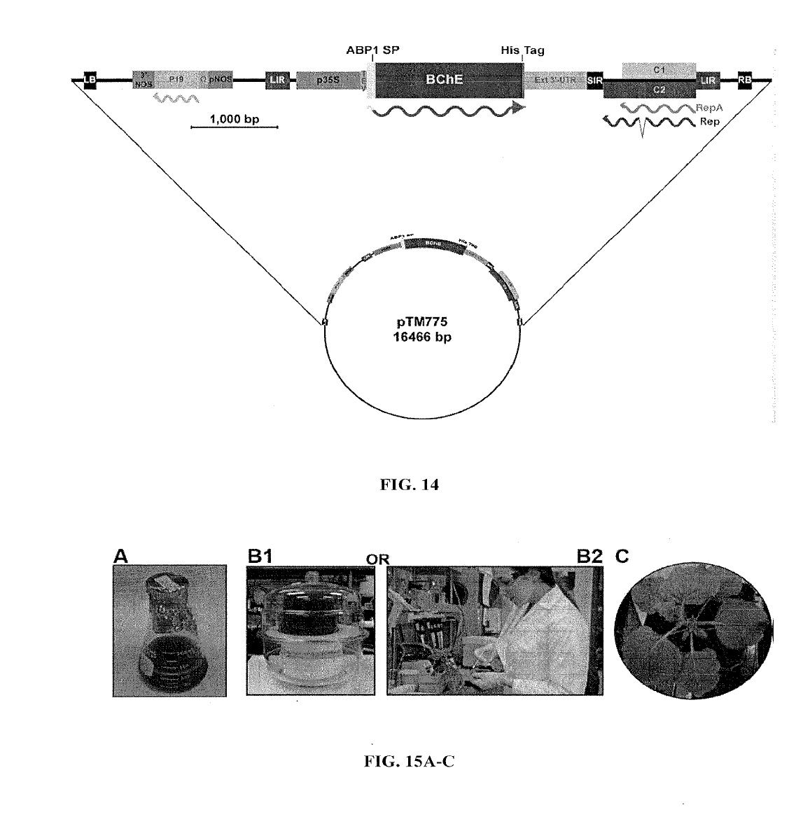

A comparison of the effectiveness of the plant-specific signal peptide and the ICON apoplastic signal peptide without the 42 amino acid extension is found in FIGS. 6 and 14. FIG. 6 demonstrates the effects of the 42 amino acid extension on expression. There was a 3.5-fold increase in the apoplastic module (˜0.016% TSP) and a 7-fold increase with the cytoplasmic module (˜0.032% TSP). Similarly, FIG. 8 demonstrates the effect of the endogenous signal peptide compared to a plant-specific signal peptide. Note the change in the axis scale for this figure. A 200-fold increase was seen with the ICON apoplastic module (˜6.6% TSP). Conversely, removal of all signal peptides and the 42 amino acid extension lead to nearly no accumulation. As seen in FIG. 9, there was 2.5-fold less expression with this system (42−/sp−/Cyto) than the original construct (42+/sp+/Cyto). A summary of the results is shown in Table 1.

TABLE 1

| | | mg BChE/kg |
|---|---|---|
| | % TSP | (fresh weight) |
| 42−/sp−/Cyto | 0.002% | 0.03 |
| 42+/sp+/Cyto | 0.005% | 0.17 |
| 42−/sp+/Apo | 0.016% | 1.04 |
| 42−/sp+/Cyto | 0.032% | 18.79 |
| 42−/sp−/Apo | 6.4% | 232.31 |

3. pTM764

Removal of the ER retention signal provided pTM764, which was another highly expressing vector (FIG. 11A; SEQ ID NOS:5 and 6). The reconstructed genome contains the following open reading frames: RdRp which encodes two subunits of the RNA-dependent RNA polymerase; MP, encoding the movement protein; plant optimized gene encoding human BChE supplied with α-amylase signal peptide and a 6 His-residue H is Tag (6×His). FIG. 11B. In the nucleic acid sequence below (SEQ ID NO:5), bold italics indicates the start codon, bold underline indicates the α-amylase signal peptide, and underlined italics indicates the His tag:

[symbol]

gcgaacaaacacttgtccctctccctcttcctcgtcctccttggcctgtcggccagcttggcctccggagccat
ggaggatgacatcatcattgccaccaagaatggtaaggttaggggtatgaacctcacagtttttggtggtactgttacag
ccttccttggtattccttatgcccaaccacctcttggtagacttaggttcaagaagccacaaagcctcaccaagtggtctg
acatttggaatgccaccaagtatgccaactcctgttgtcaaaacattgaccaatccttcccaggatttcatggatctgagat
gtggaacccaaacactgacctctctgaggattgtctttaccttaatgtgtggatcccagccccaaagcctaagaatgcca
ctgttctcatttggatctatggtggtggtttccaaactggaacctcctctctccatgtttatgatggaaagttcttggctagag
ttgagagagttattgtggtgagcatgaactatagggtgggtgccttgggattcttggccctcccaggaaatcctgaggcc
ccaggtaatatgggtctttttgaccaacaattggctcttcaatgggttcagaagaacattgctgcctttggtggaaacccta
agtctgttaccctctttggagagtctgctggagctgcttctgttagccttcacttgctttctcctggaagccactccttgttca
ctagagccattctccaatctggatccttcaatgctccttgggctgtgacatctctttatgaggctaggaatagaacattgaa
ccttgctaagttgactggttgctctagagagaatgagactgagatcatcaagtgtcttagaaacaaggacccacaagag
attcttttgaatgaggcctttgttgttccttatggaacccctttgtctgtgaactttggtcctacagtggatggtgatttcctca
ctgacatgccagacatcttgcttgagcttggacaattcaagaagacccaaattttggtgggtgttaacaaggatgagggt
acagctttccttgtgtatggcgcgcctggttttagcaaggacaacaactccatcatcactagaaaggagttccaagagg
gtctcaagatcttcttcccaggagtgtctgagtttggaaaggagtccatccttttccattacacagattgggttgatgacca
aagacctgagaactatagggaggccttgggtgatgttgttggagattacaacttcatttgccctgccttggagttcaccaa
gaagttctctgagtggggaaataatgccttcttctactactttgagcataggtcctccaagctcccttggccagagtggat
gggagtgatgcatggttatgagattgagtttgtttttggtttgcctcttgagagaagagataactacacaaaggctgagga
gatcttgagcagatccattgtgaagaggtgggccaactttgccaagtatggtaatccaaatgagactcaaaacaatagc
acaagctggcctgtgttcaagagcactgagcaaaagtacctcaccttgaacacagagtccacaaggattatgaccaag
ttgagggctcaacaatgtaggttttggacatccttcttcccaaaggtgttggagatgacaggaaatatcgatgaggctga
gtgggagtggaaggctggattccataggtggaacaactacatgatggattggaagaaccaattcaatgattacactagc
aagaaggagagctgtgtgggtctccatcaccatcaccatcactag

In the peptide sequence below (SEQ ID NO:6), bold italics indicates the alternative initiatory methionine residue, bold underline indicates the α-amylase signal peptide, and underlined italics indicates the His tag.

[symbol]

ANKHLSLSLFLVLLGLSASLASGAMEDDIIIATKNGKVRGMNLTVF
GGTVTAFLGIPYAQPPLGRLRFKKPQSLTKWSDIWNATKYANSCCQNID
QSFPGFHGSEMWNPNTDLSEDCLYNVWIPAPKPKNATVLIWIYGGGFQT
GTSSLHVYDGKFLARVERVIVVSMNYRVGALGFLALPGNPEAPGNMGLF
DQQLALQWVQKNIAAFGGNPKSVTLFGESAGAASVSLHLLSPGSHSLFT
RAILQSGSFNAPWAVTSLYEARNRTLNLAKLTGCSRENETEIIKCLRNK
DPQEILLNEAFVVPYGTPLSVNFGPTVDGDFLTDMPDILLELGQFKKTQ
ILVGVNKDEGTAFLVYGAPGFSKDNNSIITRKEFQEGLKIFFPGVSEFG
KESILFHYTDWVDDQRPENYREALGDVVGDYNFICPALEFTKKFSEWGN
NAFFYYFEHRSSKLPWPEWMGVMHGYEIEFVFGLPLERRDNYTKAEEIL
SRSIVKRWANFAKYGNPNETQNNSTSWPVFKSTEQKYLTLNTESTRIMT
KLRAQQCRFWTSFFPKVLEMTGNIDEAEWEWKAGFHRWNNYMMDWKNQF
NDYTSKKESCVGLHHHHHH

4. pTM734

The inclusion of a plant optimized BChE gene variant (Y332S) with barley α-amylase and 6×His-Tag resulted in pTM734 (FIG. 10A; SEQ ID NOS:7 and 8). The reconstructed genome contains the following open reading frames: RdRp which encodes two subunits of the RNA-dependent RNA polymerase; MP, encoding the movement protein; plant optimized gene encoding an oxime activatable variant of human BChE supplied with α-amylase signal peptide and a 6 His-residue His Tag (6×His). Site-directed mutation (Y332G) is indicated with a star. FIG. 10B. In the nucleic acid sequence below (SEQ ID NO:7), bold italics indicates the start codon, bold underline indicates the α-amylase signal peptide, underlined italics indicates the His tag, and underlined indicates Y332S.

[symbol]

gcgaacaaacacttgtccctctccctcttcctcgtcctccttggcctgtcggccagcttggcctccggagccat
ggaggatgacatcatcattgccaccaagaatggtaaggttaggggtatgaacctcacagtttttggtggtactgttacag
ccttccttggtattccttatgcccaaccacctcttggtagacttaggttcaagaagccacaaagcctcaccaagtggtctg
acatttggaatgccaccaagtatgccaactcctgttgtcaaaacattgaccaatccttcccaggatttcatggatctgagat
gtggaacccaaacactgacctctctgaggattgtctttaccttaatgtgtggatcccagccccaaagcctaagaatgcca
ctgttctcatttggatctatggtggtggtttccaaactggaacctcctctctccatgtttatgatggaaagttcttggctagag
ttgagagagttattgtggtgagcatgaactatagggtgggtgccttgggattcttggccctcccaggaaatcctgaggcc
ccaggtaatatgggtctttttgaccaacaattggctcttcaatgggttcagaagaacattgctgcctttggtggaaacccta
agtctgttaccctctttggagagtcttctggagctgcttctgttagccttcacttgctttctcctggaagccactccttgttca
ctagagccattctccaatctggttccgctaatgctccttgggctgtgacatctctttatgaggctaggaatagaacattgaa
ccttgctaagttgactggttgctctagagagaatgagactgagatcatcaagtgtcttagaaacaaggacccacaagag
attcttttgaatgaggcctttgttgttccttatggaactcctttgggagtgaactttggtcctacagtggatggtgatttcctca
ctgacatgccagacatcttgcttgagcttggacaattcaagaagacccaaattttggtgggtgttaacaaggatgagggt
acatggttccttgtgtctggagcgcctggttttagcaaggacaacaactccatcatcactagaaaggagttccaagagg
gtctcaagatcttcttcccaggagtgtctgagtttggaaaggagtccatccttttccattacacagattgggttgatgacca
aagacctgagaactatagggaggccttgggtgatgttgttggagattacaacttcatttgccctgccttggagttcaccaa
gaagttctctgagtggggaaataatgccttcttctactactttgagcataggtcctccaagctcccttggccagagtggat
gggagtgatgcatggttatgagattgagtttgtttttggtttgcctcttgagagaagagataactacacaaaggctgagga
gatcttgagcagatccattgtgaagaggtgggccaactttgccaagtatggtaatccaaatgagactcaaaacaatagc
acaagctggcctgtgttcaagagcactgagcaaaagtacctcaccttgaacacagagtccacaaggattatgaccaag
ttgagggctcaacaatgtaggttttggacatccttcttcccaaaggtgttggagatgacaggaaatatcgatgaggctga
gtgggagtggaaggctggattccataggtggaacaactacatgatggattggaagaaccaattcaatgattacactagc
aagaaggagagctgtgtgggtctccatcaccatcaccatcactag

The plant optimized BChE gene variant with enhanced cocaine hydrolytic activities contains the following amino-acid residue replacements: A199S/S287G/A328W/Y332G. BChE coding sequence is fused to barley α-amylase and a 6×His tag. pTM783, a cocaine hydrolase vector, is identical to pTM734, however the BChE sequence has the mutations described above.

In the peptide sequence below (SEQ ID NO:8), bold italics indicates the start codon, bold underline indicates the α-amylase signal peptide, underlined italics indicates the His tag, and the underlined indicates Y332S.

[symbol]

ANKHLSLSLFLVLLGLSASLASGAMEDDIIIATKNGKVRGMNLTVFGG
TVTAFLGIPYAQPPLGRLRFKKPQSLTKWSDIWNATKYANSCCQNIDQ
SFPGFHGSEMWNPNTDLSEDCLYLNVWIPAPKPKNATVLIWIYGGGFQTG
TSSLHVYDGKFLARVERVIVVSMNYRVGALGFLALPGNPEAPGNMGLFDQ
QLALQWVQKNIAAFGGNPKSVTLFGESSGAASVSLHLLSPGSHSLFTRAI
LQSGSANAPWAVTSLYEARNRTLNLAKLTGCSRENETEIIKCLRNKDPQE
ILLNEAFVVPYGTPLGVNFGPTVDGDFLTDMPDILLELGQFKKTQILVGV
NKDEGTWFLVSGAPGFSKDNNSIITRKEFQEGLKIFFPGVSEFGKESIL
FHYTDWVDDQRPENYREALGDVVGDYNFICPALEFTKKFSEWGNNAFFYY
FEHRSSKLPWPEWMGVMHGYEIEFVFGLPLERRDNYTKAEEILSRSIVKR
WANFAKYGNPNETQNNSTSWPVFKSTEQKYLTLNTESTRIMTKLRAQQCR
FWTSFFPKVLEMTGNIDEAEWEWKAGFHRWNNYMMDWKNQFNDYTSKKES
CVGLHHHHHH

A summary of the enzyme activity for pTM734 is found in Table 2.

TABLE 2
Enzyme activity U/mg protein

| | | | Average | | | Average | | | Average |
|---|---|---|---|---|---|---|---|---|---|
| | O.D | | En. | O.D | | En. | O.D | | En. |
| | 0.1 | 0.1 | Activity | 0.2 | 0.2 | Activity | 0.4 | 0.4 | Activity |
| 6 DPI | 2.58 | 1.64 | 2.11 | 1.54 | 2.04 | 1.79 | 1.58 | 1.75 | 1.665 |
| 8 DPI | 10.1 | 5.49 | 7.795 | 7.25 | 7.14 | 7.195 | 11.45 | 8.74 | 10.095 |
| 13 DPI | 29.5 | 27.51 | 28.505 | 29.87 | 37.3 | 33.585 | 21.35 | 19.53 | 20.44 |

5. pTM781 and pTM783

Vector pTM781 contains a plant optimized BChE gene variant with enhanced cocaine hydrolytic activities containing the following amino-acid residue replacements: A199S/S287G/A328W/Y332G. BChE coding sequence is fused to barley α-amylase and a 6×His tag. (SEQ ID NOS:9 and 10). In the nucleic acid sequence below (SEQ ID NO:9), bold italics indicates the start codon, bold underline indicates the α-amylase signal peptide, underlined italics indicates the His tag, and underlined indicates Y332S.

[symbol]

gcgaacaaacacttgtccctctccctcttcctcgtcctccttggcctgtcggccagcttggcctccggagccat
ggaggatgacatcatcattgccaccaagaatggtaaggttaggggtatgaacctcacagtttttggtggtactgttacag
ccttccttggtattccttatgcccaaccacctcttggtagacttaggttcaagaagccacaaagcctcaccaagtggtctg
acatttggaatgccaccaagtatgccaactcctgttgtcaaaacattgaccaatccttcccaggatttcatggatctgagat
gtggaacccaaacactgacctctctgaggattgtctttaccttaatgtgtggatcccagccccaaagcctaagaatgcca
ctgttctcatttggatctatggtggtggtttccaaactggaacctcctctctccatgtttatgatggaaagttcttggctagag
ttgagagagttattgtggtgagcatgaactatagggtgggtgccttgggattcttggccctcccaggaaatcctgaggcc
ccaggtaatatgggtctttttgaccaacaattggctcttcaatgggttcagaagaacattgctgcctttggtggaaacccta
agtctgttaccctctttggagagtcttctggagctgcttctgttagccttcacttgctttctcctggaagccactccttgttca
ctagagccattctccaatctggttccgctaatgctccttgggctgtgacatctctttatgaggctaggaatagaacattgaa
ccttgctaagttgactggttgctctagagagaatgagactgagatcatcaagtgtcttagaaacaaggacccacaagag
attcttttgaatgaggcctttgttgttccttatggaactcctttgggagtgaactttggtcctacagtggatggtgatttcctca
ctgacatgccagacatcttgcttgagcttggacaattcaagaagacccaaattttggtgggtgttaacaaggatgagggt
acatggttccttgtgtctggagcgcctggttttagcaaggacaacaactccatcatcactagaaaggagttccaagagg
gtctcaagatcttcttcccaggagtgtctgagtttggaaaggagtccatccttttccattacacagattgggttgatgacca
aagacctgagaactatagggaggccttgggtgatgttgttggagattacaacttcatttgccctgccttggagttcaccaa
gaagttctctgagtggggaaataatgccttcttctactactttgagcataggtcctccaagctcccttggccagagtggat
gggagtgatgcatggttatgagattgagtttgtttttggtttgcctcttgagagaagagataactacacaaaggctgagga
gatcttgagcagatccattgtgaagaggtgggccaactttgccaagtatggtaatccaaatgagactcaaaacaatagc
acaagctggcctgtgttcaagagcactgagcaaaagtacctcaccttgaacacagagtccacaaggattatgaccaag
ttgagggctcaacaatgtaggttttggacatccttcttcccaaaggtgttggagatgacaggaaatatcgatgaggctga
gtgggagtggaaggctggattccataggtggaacaactacatgatggattggaagaaccaattcaatgattacactagc
aagaaggagagctgtgtgggtctccatcaccatcaccatcactag

In the peptide sequence below (SEQ ID NO:10), bold italics indicates the start codon, bold underline indicates the α-amylase signal peptide, underlined italics indicates the His tag, and the underlined residues indicate A199S/F227A/S287G/A328W/Y332G, respectively.

[symbol]

ANKHLSLSLFLVLLGLSASLASGAMEDDIIIATKNGKVRGMNLTVF
GGTVTAFLGIPYAQPPLGRLRFKKPQSLTKWSDIWNATKYANSCCQNID
QSFPGFHGSEMWNPNTDLSEDCLYLNVWIPAPKPKNATVLIWIYGGGFQ
TGTSSLHVYDGKFLARVERVIVVSMNYRVGALGFLALPGNPEAPGNMGL
FDQQLALQWVQKNIAAFGGNPKSVTLFGESSGAASVSLHLLSPGSHSLF
TRAILQSGSANAPWAVTSLYEARNRTLNLAKLTGCSRENETEIIKCLRN
KDPQEILLNEAFVVPYGTPLGVNFGPTVDGDFLTDMPDILLELGQFKKT
QILVGVNKDEGTWFLVGGAPGFSKDNNSIITRKEFQEGLKIFFPGVSEF
GKESILFHYTDWVDDQRPENYREALGDVVGDYNFICPALEFTKKFSEWG
NNAFFYYFEHRSSKLPWPEWMGVMHGYEIEFVFGLPLERRDNYTKAEEI
LSRSIVKRWANFAKYGNPNETQNNSTSWPVFKSTEQKYLTLNTESTRIM
TKLRAQQCRFWTSFFPKVLEMTGNIDEAEWEWKAGFHRWNNYMMDWKNQ
FNDYTSKKESCVGLHHHHHH

### Example 2

### BeYDV-Based Vector Constructs

1. pTM580

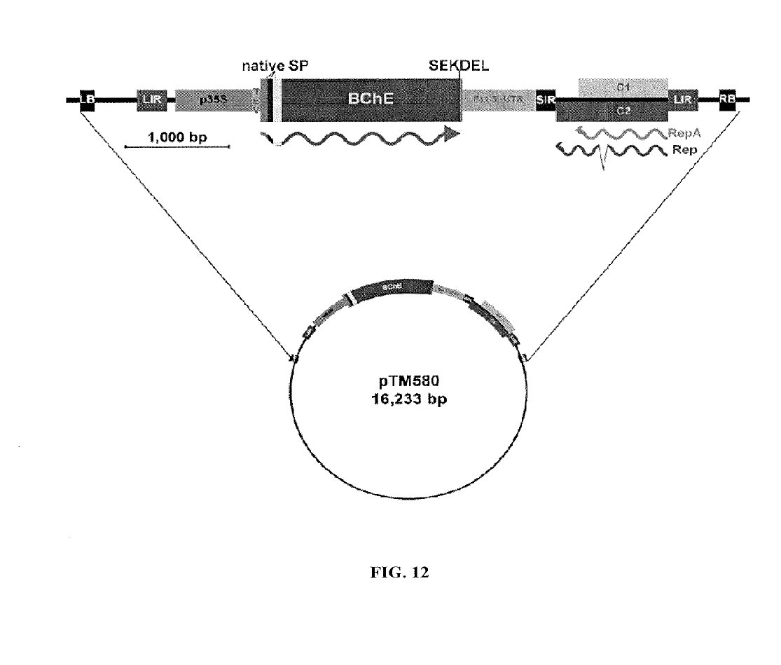

pTM580 contains a plant-optimized BChE gene with its native signal peptide, an ER retention signal, and a 42 amino acid extension (FIG. 12; SEQ ID NOS:11 and 12). In the nucleic acid sequence below (SEQ ID NO:11), bold italics indicates the alternative start codons, bold underline indicates the long endogenous signal peptide, underlined indicates the short endogenous signal peptide, and underlined italics indicates the ER retention signal.

[symbol]

ggatctgtgcaaagcaacctccaagctggagctgctgctgccagctgcatctccccaaagtactac [symbol] at
cttcactccttgcaagctctaccacctctgttgtagggagtctgagatcaac [symbol] cacagcaaggttaccatcattt
gcatcaggttcctcttttggttcctcctcctctgcatgcttattggtaagagccacactgaggatgacatcatcattgccac
caagaatggtaaggttaggggtatgaacctcacagtttttggtggtactgttacagccttccttggtattccttatgcccaa
ccacctcttggtagacttaggttcaagaagccacaaagcctcaccaagtggtctgacatttggaatgccaccaagtatg
ccaactcctgttgtcaaaacattgaccaatccttcccaggatttcatggatctgagatgtggaacccaaacactgacctct
ctgaggattgtctttaccttaatgtgtggatcccagccccaaagcctaagaatgccactgttctcatttggatctatggtgg
tggtttccaaactggaacctcctctctccatgtttatgatggaaagttcttggctagagttgagagagttattgtggtgagc
atgaactatagggtgggtgccttgggattcttggccctcccaggaaatcctgaggccccaggtaatatgggtctttttga
ccaacaattggctcttcaatgggttcagaagaacattgctgcctttggtggaaaccctaagtctgttaccctctttggaga
gtctgctggagctgcttctgttagccttcacttgctttctcctggaagccactccttgttcactagagccattaccaatctg
gatccttcaatgctccttgggctgtgacatctctttatgaggctaggaatagaacattgaaccttgctaagttgactggttg
ctctagagagaatgagactgagatcatcaagtgtcttagaaacaaggacccacaagagattcttttgaatgaggcctttg
ttgttccttatggaacccctttgtctgtgaactttggtcctacagtggatggtgatttcctcactgacatgccagacatcttgc
ttgagcttggacaattcaagaagacccaaattttggtgggtgttaacaaggatgagggtacagctttccttgtgtatggcg
cgcctggttttagcaaggacaacaactccatcatcactagaaaggagttccaagagggtctcaagatcttcttcccagg
agtgtctgagtttggaaaggagtccatccttttccattacacagattgggttgatgaccaaagacctgagaactataggg
aggccttgggtgatgttgttggagattacaacttcatttgccctgccttggagttcaccaagaagttctctgagtggggaa
ataatgccttcttctactactttgagcataggtcctccaagctcccttggccagagtggatgggagtgatgcatggttatg
agattgagtttgtttttggtttgcctcttgagagaagagataactacacaaaggctgaggagatcttgagcagatccattgt
gaagaggtgggccaactttgccaagtatggtaatccaaatgagactcaaaacaatagcacaagctggcctgtgttcaa
gagcactgagcaaaagtacctcaccttgaacacagagtccacaaggattatgaccaagttgagggctcaacaatgtag
gttttggacatccttcttcccaaaggtgttggagatgacaggaaatatcgatgaggctgagtgggagtggaaggctgga
ttccataggtggaacaactacatgatggattggaagaaccaattcaatgattacactagcaagaaggagagctgtgtgg
gtctctctgagaaggatgaactctag

In the peptide sequence below (SEQ ID NO:12), bold italics indicates the alternative initiatory methionine residues, bold underline indicates the long endogenous signal peptide, the underlined residues indicate the short endogenous signal peptide, and underlined italics indicates the ER retention signal.

[symbol]

GSVQSNLQAGAAAASCISPKYY [symbol] IFTPCKLYHLCCRESEIN [symbol]
HSKVTIICIRFLFWFLLLCMLIGKSHTEDDIIIATKNGKVRGMNLTVFG
GTVTAFLGIPYAQPPLGRLRFKKPQSLTKWSDIWNATKYANSCCQNIDQ
SFPGFHGSEMWNPNTDLSEDCLYLNVWIPAPKPKNATVLIWIYGGGFQ
TGTSSLHVYDGKFLARVERVIVVSMNYRVGALGFLALPGNPEAPGNMG
LFDQQLALQWVQKNIAAFGGNPKSVTLFGESAGAASVSLHLLSPGSHS
LFTRAILQSGSFNAPWAVTSLYEARNRTLNLAKLTGCSRENETEIIKC
LRNKDPQEILLNEAFVVPYGTPLSVNFGPTVDGDFLTDMPDILLELGQ
FKKTQILVGVNKDEGTAFLVYGAPGFSKDNNSIITRKEFQEGLKIFFP
GVSEFGKESILFHYTDWVDDQRPENYREALGDVVGDYNFICPALEFTK
KFSEWGNNAFFYYFEHRSSKLPWPEWMGVMHGYEIEFVFGLPLERRDN
YTKAEEILSRSIVKRWANFAKYGNPNETQNNSTSWPVFKSTEQKYLTL
NTESTRIMTKLRAQQCRFWTSFFPKVLEMTGNIDEAEWEWKAGFHRWN
NYMMDWKNQFNDYTSKKESCVGLSEKDEL

The pTM554 vector was used for the transient expression of BChE in Nicotiana benthamiana leaf. Considerable necrosis was seen at 4 DPI. BChE expression at 4DPI was approximately 2.77 mg/kg leaf

2. pTM771

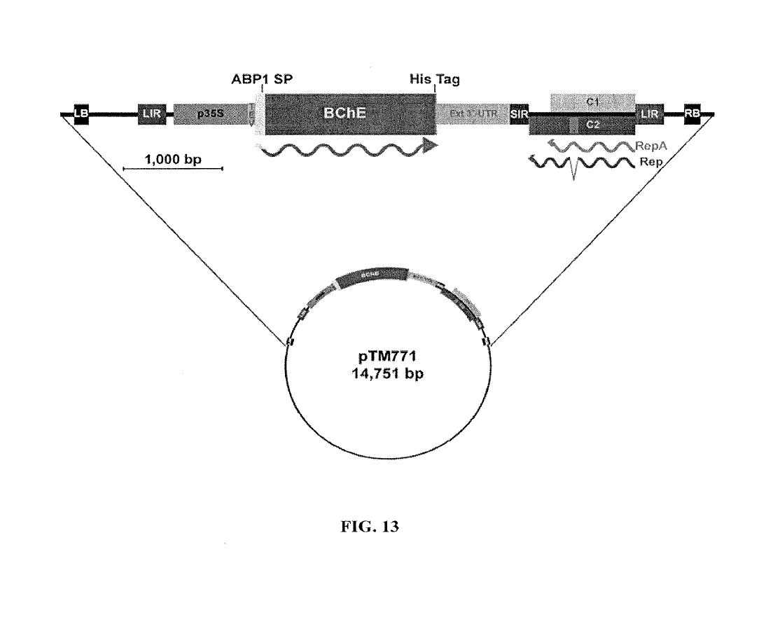

pTM771 is a WT BChE in Gemini vector. (FIG. 13; SEQ ID NOS:13 and 14) The replicon consists of duplicated Long Intergenic Regions (LIR), a Short Intergenic Region (SIR), C1/C2 open reading that encode through alternative splicing of a short intron the two BeYDV replication-associated proteins RepA and Rep (Mor et al., 2003). The BChE expression cassette contains the ³⁵S promoter of cauliflower mosaic virus with duplicated enhancer (p35S), the 5′-UTR of tobacco etch virus (TEV), the coding region of mature BChE fused to the signal peptide of tobacco auxin binding protein 1 (ABP 1 SP) on its N-terminus and a His-tag on its C-terminus (HIS Tag), followed by the 3′-UTR of the tobacco extension gene (EXT 3′-UTR) with its native signal peptide plus the ER retention signal. For details describing the pGPTV-Kan backbone see references in (Geyer et al., 2007). In the nucleic acid sequence below (SEQ ID NO:13), bold italics indicates the start codon, bold underline indicates the APB 1 signal peptide, and underlined italics indicates the His tag:

[symbol]

atcgttctttctgttggttccgcttcttcatctcctatcgtcgttgtcttttccgtggcacttcttctcttctacttct
ctgaaacttccctaggtgaggatgacatcatcattgccaccaagaatggtaaggttaggggtatgaacctcacagttttt
ggtggtactgttacagccttccttggtattccttatgcccaaccacctcttggtagacttaggttcaagaagccacaaagc
ctcaccaagtggtctgacatttggaatgccaccaagtatgccaactcctgttgtcaaaacattgaccaatccttcccagg
atttcatggatctgagatgtggaacccaaacactgacctctctgaggattgtctttaccttaatgtgtggatcccagcccca
aagcctaagaatgccactgttctcatttggatctatggtggtggtttccaaactggaacctcctctctccatgtttatgatgg
aaagttcttggctagagttgagagagttattgtggtgagcatgaactatagggtgggtgccttgggattcttggccctccc
aggaaatcctgaggccccaggtaatatgggtctttttgaccaacaattggctcttcaatgggttcagaagaacattgctg
cctttggtggaaaccctaagtctgttaccctctttggagagtctgctggagctgcttctgttagccttcacttgctttctcctg
gaagccactccttgttcactagagccattctccaatctggatccttcaatgctccttgggctgtgacatctctttatgaggct
aggaatagaacattgaaccttgctaagttgactggttgctctagagagaatgagactgagatcatcaagtgtcttagaaa
caaggacccacaagagattcttttgaatgaggcctttgttgttccttacggaactcctttgtctgtgaactttggtcctacag
tggatggtgatttcctcactgacatgccagacatcttgcttgagcttggacaattcaagaagacccaaattttggtgggtg
ttaacaaggatgagggtacagctttccttgtgtatggcgcgcctggttttagcaaggacaacaactccatcatcactaga
aaggagttccaagagggtctcaagatcttcttcccaggagtgtctgagtttggaaaggagtccatccttttccattacaca
gattgggttgatgaccaaagacctgagaactatagggaggccttgggtgatgttgttggagattacaacttcatttgccct
gccttggagttcaccaagaagttctctgagtggggaaataatgccttcttctactactttgagcataggtcctccaagctc
ccttggccagagtggatgggagtgatgcatggttatgagattgagtttgtttttggtttgcctcttgagagaagagataact
acacaaaggctgaggagatcttgagcagatccattgtgaagaggtgggccaactttgccaagtatggtaatccaaatg
agactcaaaacaatagcacaagctggcctgtgttcaagagcactgagcaaaagtacctcaccttgaacacagagtcca
caaggattatgaccaagttgagggctcaacaatgtaggttttggacatccttcttcccaaaggtgttggagatgacagga
aatatcgatgaggctgagtgggagtggaaggctggattccataggtggaacaactacatgatggattggaagaaccaa
ttcaatgattacactagcaagaaggagagctgtgtgggtctccatcaccatcaccatcactag

In the peptide sequence below (SEQ ID NO:14), bold italics indicates the initiatory methionine residue, bold underline indicates the APB 1 signal peptide, and underlined italics indicates the His tag.

[symbol]

IVLSVGSASSSPIVVVFSVALLLFYFSETSLGEDDIIIATKNGKVRG
MNLTVFGGTVTAFLGIPYAQPPLGRLRFKKPQSLTKWSDIWNATKYANS
CCQNIDQSFPGFHGSEMWNPNTDLSEDCLYLNVWIPAPKPKNATVLIWI
YGGGFQTGTSSLHVYDGKFLARVERVIVVSMNYRVGALGFLALPGNPEA
PGNMGLFDQQLALQWVQKNIAAFGGNPKSVTLFGESAGAASVSLHLLSP
GSHSLFTRAILQSGSFNAPWAVTSLYEARNRTLNLAKLTGCSRENETEI
IKCLRNKDPQEILLNEAFVVPYGTPLSVNFGPTVDGDFLTDMPDILLEL
GQFKKTQILVGVNKDEGTAFLVYGAPGFSKDNNSIITRKEFQEGLKIFF
PGVSEFGKESILFHYTDWVDDQRPENYREALGDVVGDYNFICPALEFTK
KFSEWGNNAFFYYFEHRSSKLPWPEWMGVMHGYEIEFVFGLPLERRDNY
TKAEEILSRSIVKRWANFAKYGNPNETQNNSTSWPVFKSTEQKYLTLNT
ESTRIMTKLRAQQCRFWTSFFPKVLEMTGNIDEAEWEWKAGFHRWNNYM
MDWKNQFNDYTSKKESCVGLHHHHHH

### Example 3

### Plants as a Source of Butyrylcholinesterase Variants Designed for Enhanced Cocaine Hydrolase Activity

Cloning of Plant-Expression Optimized Synthetic Genes Encoding BChE Variants and their Expression in Plants.

The plant-expression optimized gene encoding the WT form of human BChE, pBChE (Geyer et al., 2009; Geyer et al., 2010) with C-terminal His-tag (H6) was used as template for introduction of site-directed mutations (QuickChange kit, Stratagene) to create the following sited-directed mutations: F227A/S287G/A328W/Y332A, A199S/S287G/A328W/Y332G (Yang et al., 2010), A199S/F227A/S287G/A328W/Y332G, and F227A/S287G/A328W/Y332G) (Zheng et al., 2010). The genes were transiently expressed in wild-type (WT) N. benthamiana plants using the MagnICON vector system based on deconstructed tobacco mosaic virus (Santi et al., 2006).

### Enrichment Preparation of BChE Variants and Biochemical Analyses

The proteins were partially purified following a protocol similar to one used for WT pBChE (Geyer et al., 2009; Geyer et al., 2010) based on concanavalin A (ConA) chromatography.

Estimation of concentration of BChE and variants thereof was conducted using quantitative immunoblot assay with highly purified samples of plasma-derived and plant-derived BChE, whose molar concentrations were previously determined (Geyer et al., 2009; Geyer et al., 2010) serving as standards. To this end, standards were resolved by SDS-PAGE on 8% polyacrylamide gels, transferred to nitrocellulose membranes, immunodecorated with rabbit polyclonal anti-hBChE antibodies, and detected by anti-rabbit IgG-Horse Radish Peroxidase (HRP) antibodies followed by chemiluminescence assay. High resolution (at least 600 dpi) greyscale images were used for densitometry analysis with Image J Software and data was used to plot standard curves fitted by linear regression (GraphPad Prism). Samples of variants with unknown concentrations were resolved alongside the standards and densitometry results together with the regression equations were used to obtain concentration of the BChE variants. Several dilutions of samples were applied to make sure samples were well within the linear range of the standard curve. Results showed excellent correlation with butyrylthiocholine (BTC) hydrolysis assays (see below) by the mutants and individual specific activities could thus be calculated. In all subsequent experiments the inventors have used these specific activities to estimate BChE variant concentration.

### Enzymatic Assays

Two enzyme assays were performed. The spectrophotometric Ellman assay was used to assess basic BChE activity with BTC (Sigma) as the substrate (1 mM). Assays were run at 30° C. in a Spectramax 190 spectrophotometer (Molecular Devices) as previously described (Geyer et al., 2005). To evaluate cocaine hydrolysis, a previously described radiometric assay was used with 3H cocaine as substrate over a wide range of concentrations (Brimijoin et al., 2002). Data were subjected to non-linear regression analysis (Sigma-Plot), and estimates of VMAX and KM were derived along with their standard errors. Turnover numbers (KCAT) could be derived, in turn, from these Vmax values and the assay's molar concentrations of BChE variants obtained as described above.

### Results

The BChE variants are as follows: Variant 1 (BChE A328W/Y332A), Variant 2 (F227A/S287G/A328W/Y332A), Variant 3 (A199S/S287G/A328W/Y3326), Variant 4 (A199S/F227A/S287G/A328W/Y332G), and Variant 5 (F227A/S287G/A328W/Y332G).

Using the MagnICON expression system (Santi et al., 2006), deconstructed-TMV-based vectors were introduced into WT tobacco plants by infiltration either by using needle-less syringe injection or by application of vacuum on whole plants submerged in agrobacterial suspensions (FIG. 15).

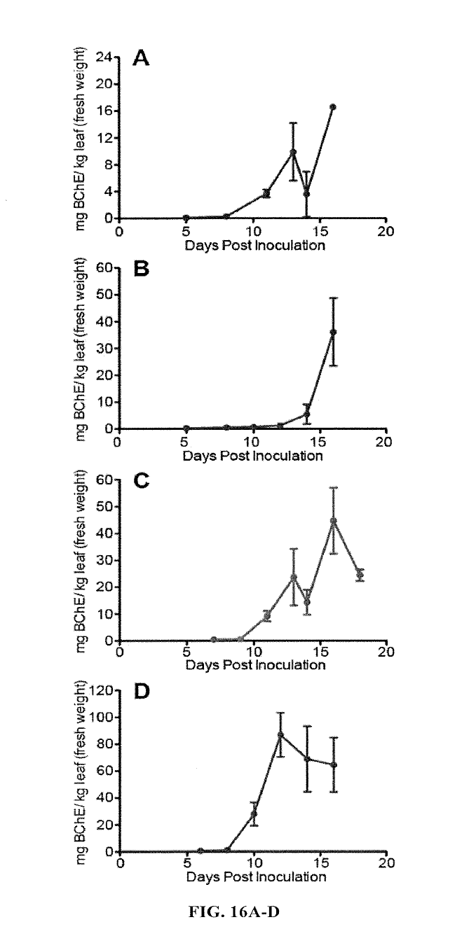

Leaf samples were harvested at the indicated time points and assayed by the Ellmanassay and immunoassay to determine the expression level of the BChE enzyme variants (FIG. 2). Multiple 0.2 g leaf samples from different plants were assayed per time point for BChE activity. Peak expression time was around 14 days but with some variation among the variant foams (14-17 days). Accumulation levels varied considerably between the various mutants and ranged from 16 to 100 mg per kg fresh weight leaf material (FIG. 16).

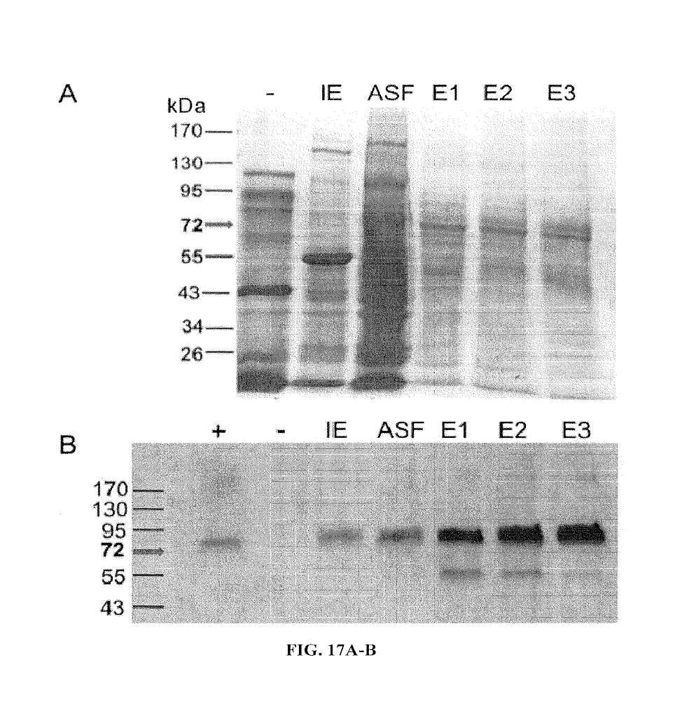

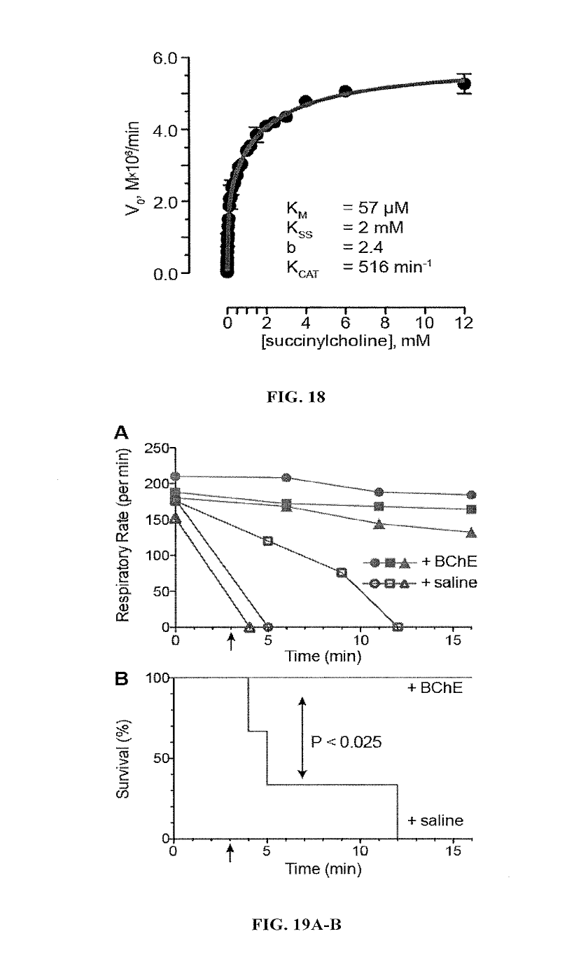

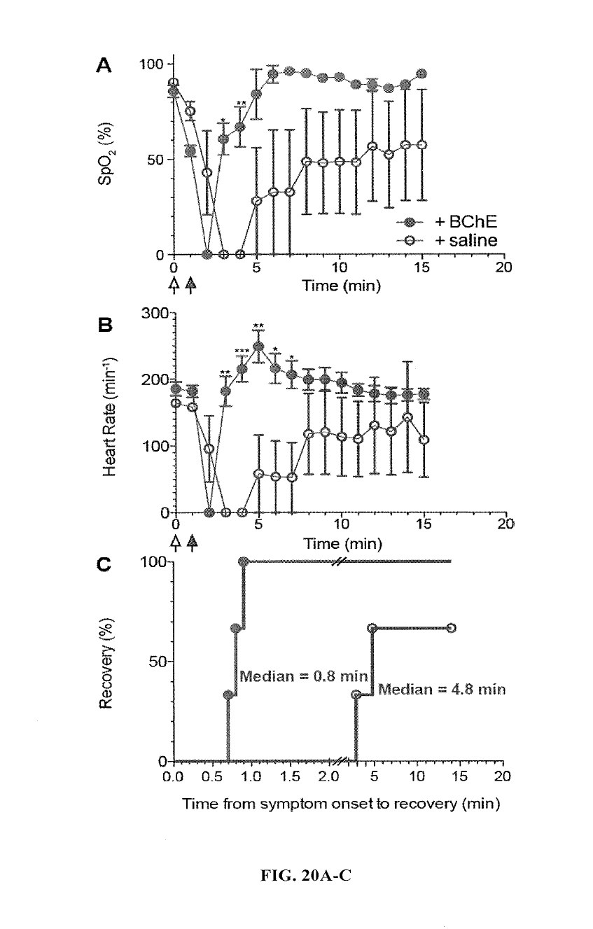

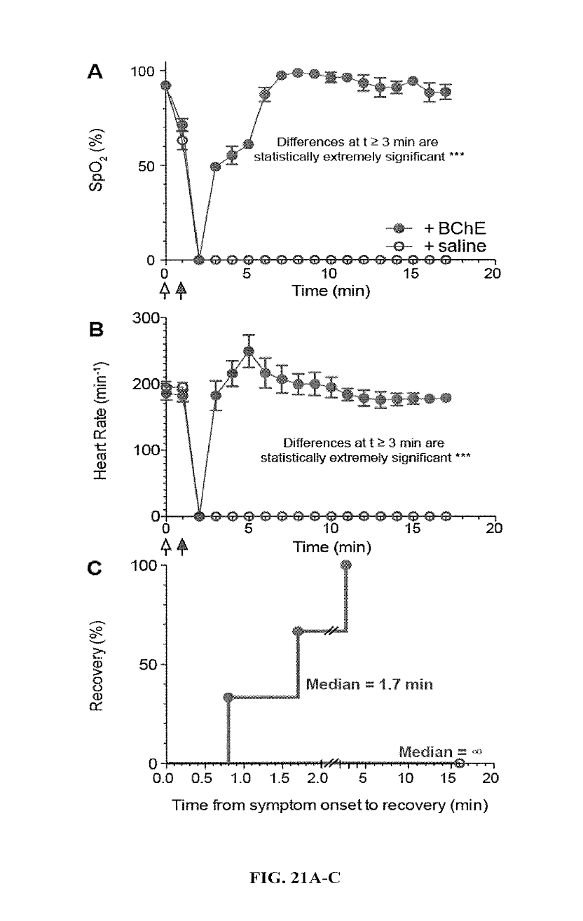

Partial purification was achieved by ConA affinity chromatography as exemplified for Variant 4 (FIG. 17), and Variants 3-5 were tested for cocaine hydrolysis activity in a radiometric assay (Brimijoin et al., 2002). Michaelis-Menten constant (KM) values for Variants 3-5 were (mean±SEM, respectively) 2.6±0.1, 2.7±0.1, and 12.4±1.2 μM compared to the reported WT BChE KM of 4.5 μM (Sun et al., 2002). The turnover number was determined for one variant thus far (Variant 4) and was 5200±63 min-1 (mean±SEM), similar to the established value of 5700 min-1 determined for the variant derived from mammalian cell system (Zheng et al., 2008). The efficiency of catalysis (KCAT/KM) was determined for one mutant thus far (Variant 4) and was (1.91±0.09)×109 M·min-1, a ˜1500 fold increase over the established value of 1.3×106 M·min-1 for WT BChE [4]. This outcome is very similar to that reported for the original version of the same mutant expressed in mammalian cell culture (Zheng et al., 2008).

These results show that Nicotiana benthamiana can be used to express different cocaine hydrolase variants of BChE, including Variant 4 (A199S/F227A/S287G/A328W/Y332G), which is the most efficient cocaine hydrolyzing variant of BChE designed to date (Yang et al., 2010, Zheng et al., 2010). Average peak projected yield was found to range from 16-100 mg BChE/kg fresh weight leaf material. Of those plant-derived variants tested, all have been found to exhibit nearly identical kinetic properties to those variants derived from other sources.

### Example 4

### Reversal of Succinylcholine Induced Apnea with an Organophosphate Scavenging Recombinant Butyrylcholinesterase

### Preparation of Recombinant Butyrylcholinesterase

Transgenic plants expressing a plant-optimized synthetic gene encoding BChE were created as previously described (Geyer et al., 2010). Briefly, stable Nicontiana benthamiana lines expressing a codon-optimized human butyrylcholinesterase were created and screened for maximal expression. The lines with highest accumulation were expanded from homozygous seed stocks and propagated under greenhouse conditions. Plant-derived BChE (pBChE) was prepared from mature 8-11 week old plants that were juiced in the presence of 150 mM sodium metabisulfite, and the juice was strained and clarified by centrifugation. The 30%-70% ammonium sulphate fraction (pH 4.0) was resuspended and subjected at in-tandem affinity chromatography through Concanavalin A-Sepharose 4B and then procainamide-agarose gel custom resin. Eluate was serial dialyzed against 0.125× phosphate-buffered saline (PBS), pH 7.4, then concentrated and stored with 0.02% azide at 4° C. for up to 6 months. Prior to use, the preparation was dialyzed again to remove azide.

### Biochemical Analysis

Assay of butyrylcholine hydrolysis followed the method of Ellman as described in (Geyer et al., 2010). Succinylcholine hydrolase activity was monitored by the method of George and co-workers (George et al., 1988) with modifications to fit a 96-well plate format. Briefly, our standard succinylcholine-hydrolysis buffer contained 100 mM NaH₂PO₄/Na₂HPO₄ buffer pH 7.5, 0.77 mM phenol, 0.15 mM 4-aminoantipyrine, 1 U/mL choline oxidase, and 1.2 U/mL horse raddish peroxidase type I. Appropriate volumes of 10× stock solutions (in 100 mM NaH₂PO₄/Na₂HPO₄ buffer pH 7.5) were pre-mixed and dispensed at 160 μL aliquotes onto 96-well plates followed by addition of the substrate succinylcholine chloride (20 μl, final concentrations as indicated). Reactions were started by addition of pBChE (4.74 nM) to yield a final well volume of 200 μl. Hydrolysis was monitored by recording absorbance changes at 500 nm. Self hydrolysis rates were measured on samples that contained no enzyme and were subtracted from the enzymatically catalyzed reaction rates. A choline standard curve was similarly created by using the same assay except that choline chloride replaced succinylcholine (final concentration range of 10-100 μM).

Kinetic analysis was done according to Radic et al. (Radic et al., 1993) as follows. Initial enzyme velocity, V₀, was plotted as a function of substrate concentration and the results were fitted by nonlinear regression using GraphPad Prism to the following equation:

$${v}_{0} = ( \frac{1 + b [ SC ] / {K}_{SS}}{1 + [ SC ] / {K}_{SS}} ) ( \frac{{V}_{max}}{1 + {K}_{M} / [ SC ]} )$$

where [SC] is the concentration of SC, Vₘₐₓ is maximal velocity, K_{M} is the Michaelis-Menten constant, K_{SS} is the dissociation constant of substrate from the enzyme's peripheral binding site, and b a factor that reflect the efficiency of hydrolysis of the substrate in the presence of another substrate molecule bound at the peripheral site (with substrate activation when b>1).

### In Vivo Experiments

Mouse experiments were conducted as follows. Male FVB/N mice (Mus musculus, 8-12 weeks old) were anesthetized by injection of ketamine/xylazine/acepromazide cocktail at the dose of 0.05 mL per 25 g of total body weight (concentrations were, respectively 21 mg/mL, 2.4 mg/mL, and 0.3 mg/mL). Anesthetized mice were assessed for respiratory rate by counting respirations for 30 seconds using a Littman model 3000 electronic stethoscope placed on the mouse left mid axillary line and extrapolating the per minute rate. Mice were then injected intravenously (tail vein) with 1 mg/kg succinylcholine (time 0 min). At time+3 min mice were injected with pBChE (0.6 mg/kg, ˜15 Upper animal) in 0.9% saline (or 0.9% saline vehicle control) and respiratory rate was obtained as above at the indicated time points. At time+15 minutes all surviving mice were euthanized by CO₂ asphyxiation and subsequent cervical dislocation. At no time during the experiment did the mice receive any other therapy including, but not limited to, airway protection/management, artificial ventilations, compressions, or any pharmacological assistance.

Guinea pig experiments were conducted as follows. Male Hartley guinea pigs (Cavia porcellus, 8 weeks old) were anesthetized with 90 mg/kg ketamine and 10 mg/kg xylazine. Once anesthetized, baseline heart rate (beats per minute) and Sp0₂ (%) were obtained using a Surgivet Plus Veterinary Anesthesia and Monitoring Module, model #V3404. Guinea pigs were then injected intravenously (leg vein) with 0.334 mg/kg SC (time 0). Heart rate and Sp0₂ were obtained every minute throughout the course of the experiment. At time+1 min, groups of three apneic guinea pigs were injected with either pBChE (0.19 mg/kg, ˜48 Upper animal) in 0.9% saline (or 0.9% saline vehicle control). At time+15 minutes all surviving guinea pigs were euthanized by CO₂ asphyxiation and subsequent cervical dislocation. As above, guinea pigs received no additional care or therapy during experimentation.

### Statistical Analyses

Statistical analyses were carried out using the GraphPad Prism software. Log-rank (Mantel-Cox) test was used to determine significance of the difference between survival curves.

Statistical significance of differences between means of heart rate and Sp0₂ was tested using 1-way analysis of variance (ANOVA) followed by Bonferronits Multiple Comparison Test.

### Results

Initial characterization of plant-derived, recombinant human butyrylcholinesterase (pBChE) was previously published and was found to be invariable from that of the human plasma-derived enzyme in its ability to interact with its acetylcholine and butyrylcholine substrates and various inhibitors (Geyer et al., 2010), (Geyer et al., 2010), (Geyer et al., 2008). These studies included detailed in-vitro and in-vivo demonstration of the ability of the plant-derived enzyme to scavenge organophosphate pesticides and nerve-agents (“nerve gasses”). Here the inventors extend these studies in order to investigate the potential of pBChE to reverse SC-induced apnea and therefore determined its SC hydrolytic capacity. Succinylcholine hydrolysis by pBChE proceeded in a linear time-dependent manner with BChE (data not shown) allowing us to calculate the initial enzyme velocity (V₀). Conducting the experiment at increasing SC concentrations and plotting the V₀ values as a function of the SC concentration (FIG. 1) allowed us to obtain the Michaelis constant (K_{M}=57±7 μM) and the turnover number (K_{cat}=516±33 min⁻¹, FIG. 1). The catalytic efficiency (K_{CAT}/K_{M}) was calculated to be 9×10⁶ M⁻¹ min⁻¹. These values were consistent with previously published results for human BChE (K_{M}=35 μM and K_{cat}=600 min⁻¹) (Lockridge, 1990). Differences might be attributed to the difference in the assay used in the published research that employed an SA thioester analog. As is the case with many other substrates of BChE, the enzyme fail to reach saturation and is undergoing substrate activation, presumably due to allosteric interactions involving the peripheral substrate binding site.

To test our study's hypothesis that pBChE could reverse succinylcholine-induced apnea, the inventors turned to two distinct species animal-models. Initial studies were conducted with mice that were intravenous administered with 1 mg/kg SC, a dose which constitute about >3×LD₅₀ (0.28 mg/kg) (Lewis, 1996) and proved to be lethal to 100% of tested animals. Mice were then randomized to receive either 15 U BChE or vehicle control (0.9% saline) at 3 min following SC injection. Respiratory rate was monitored every five minutes following SC injection. While all three mice receiving SC+0.9% saline succumbed to the SC-induced respiratory depression and subsequently died, all three mice receiving SC+15 U BChE survived (FIG. 2).

To further understand the effect and kinetics of BChE-mediated SC detoxification using continuous vital sign monitoring, the inventors moved to a larger rodent model of SC toxicity. Utilizing guinea pigs, the inventors intravenously injected groups of three animals with 0.334 mg/kg (an LD₁₀₀). Complete apnea and resultant decreases in oxygen saturation were seen in both groups at 1 min following SC injection, at which time study animals received either 48 U BChE or vehicle control (0.9% saline) (FIG. 3a). Animals in both groups went on to demonstrate an absence of measurable pulse and oxygen saturation at 2 min following SC injection (1 min post BChE) (FIG. 3a, b). At 3 min following SC injection (2 min following BChE or control), guinea pigs receiving BChE had a return of spontaneous respirations, venous oxygenation had risen to 49% and heart rate was at baseline. The inventors did witness a fairly consistent response of post-anoxic tachycardia in recovering guinea pigs at 5 min post SC injection, however by 7 min post SC injection all vital signs had returned to baseline in this group. At no point following loss of measurable pulse and venous oxygen saturation did these signs return in animals receiving SC 0.9% saline.

### Example 5

### Truncation of the Terminal Residues

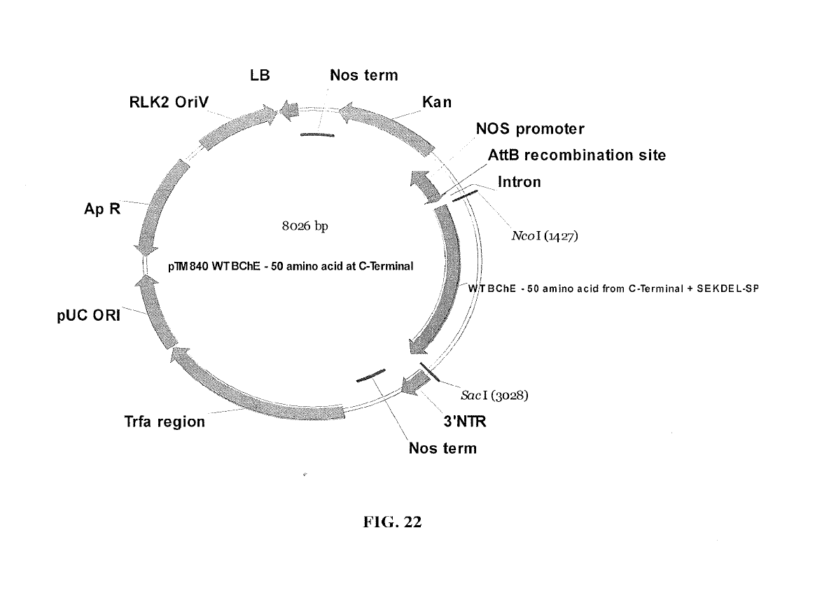

A synthetic, plant-expression optimized genes directing the synthesis of monomeric forms of human BChE will be constructed. One variant will have the cysteine residue at position 571 replaced with an alanine residue (designated as C571A). This cysteine is involved in interchain disulfide bridge formation leading to dimerization of the BChE protein, and its elimination results in mostly a monomeric variant on the expense of elimination of the dimeric form and almost complete elimination of the tetrameric forms. Dimerization is not a precondition for tetramerization, but covalent links within each of the two dimers that make up the tetramer greatly stablize the latter (see Blong et al., 1997). A more complete monomerization can be achieved by deletion of the whole oligomerization domain which is comprised of the 40 C-terminal residues (434 to 574; designated as A534-574). In the nucleic acid sequence below (pTM 840; FIG. 22; SEQ ID NO:15), bold italics indicates the start codon and underlined italics indicates SEKDEL (ER retention signal):

[symbol]

gaggatgacatcatcattgccaccaagaatggtaaggttaggggtatgaacctcacagtttttggtggtactgttac
agccttccttggtattccttatgcccaaccacctcttggtagacttaggttcaagaagccacaaagcctcaccaagtggtc
tgacatttggaatgccaccaagtatgccaactcctgttgtcaaaacattgaccaatccttcccaggatttcatggatctga
gatgtggaacccaaacactgacctctctgaggattgtctttaccttaatgtgtggatcccagccccaaagcctaagaatg
ccactgttctcatttggatctatggtggtggtttccaaactggaacctcctctctccatgtttatgatggaaagttcttggcta
gagttgagagagttattgtggtgagcatgaactatagggtgggtgccttgggattcttggccctcccaggaaatcctgag
gccccaggtaatatgggtctttttgaccaacaattggctcttcaatgggttcagaagaacattgctgcctttggtggaaac
cctaagtctgttaccctctttggagagtctgctggagctgcttctgttagccttcacttgctttctcctggaagccactccttg
ttcactagagccattctccaatctggatccttcaatgctccttgggctgtgacatctctttatgaggctaggaatagaacatt
gaaccttgctaagttgactggttgctctagagagaatgagactgagatcatcaagtgtcttagaaacaaggacccacaa
gagattcttttgaatgaggcctttgttgttccttatggaacccctttgtctgtgaactttggtcctacagtggatggtgatttcc
tcactgacatgccagacatcttgcttgagcttggacaattcaagaagacccaaattttggtgggtgttaacaaggatgag
ggtacagctttccttgtgtatggcgcgcctggttttagcaaggacaacaactccatcatcactagaaaggagttccaaga
gggtctcaagatcttcttcccaggagtgtctgagtttggaaaggagtccatccttttccattacacagattgggttgatgac
caaagacctgagaactatagggaggccttgggtgatgttgttggagattacaacttcatttgccctgccttggagttcacc
aagaagttctctgagtggggaaataatgccttcttctactactttgagcataggtcctccaagctcccttggccagagtgg
atgggagtgatgcatggttatgagattgagtttgtttttggtttgcctcttgagagaagagataactacacaaaggctgag
gagatcttgagcagatccattgtgaagaggtgggccaactttgccaagtatggtaatccaaatgagactcaaaacaata
gcacaagctggcctgtgttcaagagcactgagcaaaagtacctcaccttgaacacagagtccacaaggattatgacca
agttgagggctcaacaatgtaggttttggacatcctctgagaaggatgaactctag

These two variants retain their enzymatic and inhibitor binding properties (see Blong et al., 1997), but are expected to have a shorter time to maximal serum concentration when delivered intramuscularly (i.m.). Similar truncation was shown in a monomeric form of AChE.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

U.S. Pat. No. 4,683,202
U.S. Pat. No. 5,302,523
U.S. Pat. No. 5,322,783
U.S. Pat. No. 5,384,253
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,928,906
U.S. Pat. No. 5,945,100
U.S. Pat. No. 5,981,274
U.S. Pat. No. 5,994,624
Aharoni et al., Proc. Natl. Acad. Sci. USA, 101(2):482-487, 2004.
Ashani, Drug Dev. Res., 50(3-4):298-308, 2000.
Ausubel et al., Current Protocols in Molecular Biology, Greene Publishing Associates and Wiley Interscience, N.Y., 1994.
Baldassarre et al., Reprod. Fertil. Dev., 16(4):465-470, 2004.
Belanger et al., Faseb. J., 14(14):2323-2328, 2000.
Blong et al., The Biochemical Journal 327 (3): 747-757, 1997.
Brennan et al., J. Virol., 73(2):930-938, 1999.
Brennan et al., Mol. Biotechnol., 17(1):15-26, 2001.
Brimijoin et al., Analytical Biochemistry 309: 200-205, 2002.
Broomfield et al., Chem. Biol. Interact., 119-120:413-418, 1999.
Carbonelli et al., FEMS Microbiol. Lett., 177(1):75-82, 1999.
Cerasoli et al., Chem. Biol. Interact., 157-158:362, 2005.
Chan et al., J. Biol. Chem., 273(16):9727-9733, 1998.
Chen and Okayama, Mol. Cell. Biol., 7(8):2745-2752, 1987.
Cocea, Biotechniques, 23(5):814-816, 1997.
Cohen et al., J. Mol. Neurosci., 21(3):199-212, 2003.
Dalsgaard et al., Nat. Biotechnol., 15(3):248-252, 1997.
Doctor and Saxena, Chem. Biol. Interact., 157-158:167-171, 2005.
Durrani et al., J. Immunol. Methods, 220(1-2):93-103, 1998.
Farchi et al., J. Physiol., 546(Pt 1):165-173, 2003.
Fechheimer et al., Proc Natl. Acad. Sci. USA, 84:8463-8467, 1987.
Fernandez-Fernandez et al., Virology, 280(2):283-291, 2001.
Fletcher et al., Plant Mol. Biol., 2004; 55(1):33-43, 2004.
Fraley et al., Proc. Natl. Acad. Sci. USA, 76:3348-3352, 1979.
Franconi et al., Cancer Res., 62(13):3654-3658, 2002.
Friedman et al., Nat. Med., 2(12):1382-1385, 1996.
Gao et al., Mol Pharmacol 75:318-323, 2009.
George et al., Clin Biochem 21(3):159-162, 1988.
Geyer et al., Chem. Biol. Interact., 157-158:331-334, 2005.
Geyer et al., Proc Natl Acad Sci USA, 107(47):20251-20256, 2010.
Geyer B C, Kannan L, Chemi I, Woods R R, Soreq H, Mor T S: Transgenic plants as a source for the bioscavenging enzyme, human butyrylcholinesterase Plant Biotechnol J 2010, 8(8):873-886.
Geyer B C, Woods R R, Mor T S: Increased organophosphate scavenging in a butyrylcholinesterase mutant. Chem Biol Interact 2008, 175(1-3):376-379.
Geyer et al., C. G. Ramesh, (Ed.), Handbook of Toxicology of Chemical Warfare Agents, Academic Press, San Diego, pp. 691-717, 2009.
Geyer et al., Plant Biotechnol J, 8:873-886, 2010.
Gilleland et al., FEMS Immunol. Med. Microbiol., 27(4):291-297, 2000.
Gleba et al., Curr. Opin. Plant Biol., 7(2):182-188, 2004.
Gleba et al., Vaccine, 23(17-18):2042-2048, 2005.
Gopal, Mol. Cell Biol., 5:1188-1190, 1985.
Graham and Van Der Eb, Virology, 52:456-467, 1973.
Greenfield et al., Neuroscience, 113(3):485-492, 2002.
Grisaru et al., Eur. J. Biochem., 264(3):672-686, 1999.
Grunwald et al., J. Biochem. Biophys. Methods, 34(2):123-135, 1997.
Gunderson et al., Neurology, 42(5):946-950, 1992.
Harel et al., Nat. Struct. Mol. Biol., 11(5):412-419, 2004.
Harland and Weintraub, J. Cell Biol., 101(3):1094-1099, 1985.
Joelson et al., J. Gen. Virol., 78(Pt 6):1213-1217, 1997.
Kaeppler et al., Plant Cell Rep., 9:415-418, 1990.
Kaliste-Korhonen et al., Hum. Exp. Toxicol., 15(12):972-978, 1996.
Kaneda et al., Science, 243:375-378, 1989.
Kato et al, J. Biol. Chem., 266:3361-3364, 1991.
Kaufer et al., Nature, 393(6683):373-377, 1998.
Kronman et al., Gene, 121(2):295-304, 1992.
Lee, Jama, 290(5):659-662, 2003.
Lenz et al., Chem. Biol. Interact., 157-158:205-210, 2005.
Levenson et al., Hum. Gene Ther., 9(8):1233-1236, 1998.
Lev-Lehman et at, J. Mol. Neurosci., 14(1-2):93-105, 2000.
Lewis R J: Sax's Dangerous Properties of Industrial Materials., 9th ed edn. New York, N.Y.: Van Nostrand Reinhold; 1996.
Li et al., Biochem. Pharmacol., 70(11):1673-1684, 2005.
Liu et al., Annual Report: John Innes Center; 1998, 1998.
Lockridge et al., Biochemistry, 36(4):786-795, 1997.
Lockridge, Pharmacol Ther 47(1):35-60, 1990.
Maniatis, et al., Molecular Cloning, A Laboratory Manual, Cold Spring Harbor Press, Cold Spring Harbor, N.Y., 1988.
Marillonnet et al., Nat. Biotechnol., 23(6):718-723, 2005.
Marillonnet et al., Proc. Natl. Acad. Sci. USA, 101(18):6852-6857, 2004.
Marrs, Pharmacol. Ther., 58(1):51-66, 1993.
Marusic et al., J. Virol., 75(18):8434-8439, 2001.
Massoulie et al., Chem. Biol. Interact., 119-120:29-42, 1999.
McCormick et al., Proc. Natl. Acad. Sci. USA, 96(2):703-708, 1999.
McInerney et al., Vaccine, 17(11-12):1359-1368, 1999.
Meshorer et al., Science, 295(5554):508-512, 2002.
Millard and Broomfield, J. Neurochem., 64(5):1909-1918, 1995.
Millard et al., Biochemistry, 34(49):15925-15933, 1995.
Millard et al., Biochemistry, 37(1):237-247, 1998.
Mor and Soreq, In: Human cholinesterases from Plants for detoxification, Goodman (Ed.), Encyclopedia of Plant and Crop Science, NY, Marcel Dekker, Inc., 564-567, 2004.
Mor et al., Biotechnol. Bioeng., 75(3):259-266, 2001.
Mor et al., Biotechnol. Bioeng., 81(4):430-437, 2003.
Nagao et al., Toxicol. Appl. Pharmacol., 144(1):198-203, 1997.
Natilla et al., Arch. Viral., 149(1):137-154, 2004.
Nicolau and Sene, Biochim. Biophys. Acta, 721:185-190, 1982.
Nicolau et al., Methods Enzymol., 149:157-176, 1987.
O'Brien et al., Virology, 270(2):444-453, 2000.
Palmer and Rybicki, Plant Sci., 129:115-130, 1997.
PCT Appln. WO 94/09699
PCT Appln. WO 95/06128
Potrykus et al., Mol. Gen. Genet., 199(2):169-177, 1985.
Radio et al., Biochemistry, 32(45):12074-12084, 1993.
Rippe, et al., Mol. Cell. Biol., 10:689-695, 1990.
Sambrook et al., In: Molecular cloning: a laboratory manual, 2^{nd} Ed., Cold Spring Harbor Laboratory Press, Cold Spring Harbor, N.Y., 1989.
Santi et al., Proc. Natl. Acad. Sci. USA, 103(4):861-866, 2006.
Scholthof et al., Annu. Rev. Phytopathology, 34:299-323, 1996.
Schwarz et al., Pharmacol. Ther., 67(2):283-322, 1995.
Shohami et al., J. Mol. Med., 78(4):228-236, 2000.
Soreq and Seidman, Nat. Rev. Neurosci., 2(4):294-302, 2001.
Sultatos, J. Toxicol. Environ. Health, 43(3):271-289, 1994.
Taylor and Radic, Annu. Rev. Pharmacol. Toxicol., 34:281-320, 1994.
Taylor, In: Anticholinesterase Agents, Hardman et al., (Eds.), Goodman & Gilman's The Pharmacological Basis of Therapeutics, 9^{th} Ed., NY, McGraw-Hill, 161-76, 1996.
Timmermans et al., Ann. Rev. Plant Physiol. Plant Mol. Biol., 45:79-112, 1994.
Turpen, Philos. Trans. R Soc. Lond. B Biol. Sci., 354(1383):665-673, 1999.
Velan et al., Cell Mol. Neurobiol., 11(1):143-156, 1991.
Wong et al., Gene, 10:87-94, 1980.
Yang et al., Chemico-biological interactions 187:148-152, 2010.
Yamasue et al., Proc. Natl. Acad. Sci. USA, 100(15):9039-9043, 2003.
Yusibov et al., Proc. Natl. Acad. Sci. USA, 94(11):5784-5788, 1997.
Zhang et al., Biotechnol. Bioeng., 93(2):271-279, 2006.
Zheng et al., Journal of the American Chemical Society, 130:12148-12155, 2008.
Zheng et al., Biochemistry 49:9113-9119, 2010.

1. A viral vector containing a plant codon-optimized DNA sequence that results in accumulation of a cholinesterase in a plant leaf at levels greater than 20 mg of the enzyme per kilogram of the plant leaf.

2. The viral vector of claim 1, wherein the level of accumulation of the cholinesterase is greater than 50 mg of the enzyme per kilogram of the plant leaf.

3. The viral vector of claim 2, wherein the level of accumulation of the cholinesterase is greater than 200 mg of the enzyme per kilogram of the plant leaf.

4. The viral vector of claim 1, wherein the level of accumulation of the cholinesterase is between 200 mg and 500 mg of the enzyme per kilogram of the plant leaf.

5. The viral vector of claim 1, wherein the viral vector is derived from a tobamovirus.

6. The viral vector of claim 1, wherein the viral vector is derived from a geminivirus.

7. The viral vector of claim 6, wherein the geminivirus is a Bean Yellow Dwarf geminivirus.

8. The viral vector of claim 1, wherein the viral vector contains a non-native signal peptide.

9. The viral vector of claim 8, wherein the non-native signal peptide is a plant signal sequence.

10. The viral vector of claim 8, wherein the non-native signal peptide is a synthetic signal sequence.

11. The viral vector of claim 10, wherein the synthetic signal peptide controls plant cell localization of the cholinesterase.

12. The viral vector of claim 1, wherein the cholinesterase is acetylcholinesterase.

13. The viral vector of claim 12, wherein the plant codon-optimized DNA sequence encodes an amino acid sequence that is at least 90% identical to an amino acid sequence which corresponds to a human acetylcholinesterase.

14. The viral vector of claim 13, wherein the plant codon-optimized DNA sequence encodes an amino acid sequence that is at least 95% identical to an amino acid sequence which corresponds to a human acetylcholinesterase.

15. The viral vector of claim 14, wherein the plant codon-optimized DNA sequence encodes a human acetylcholinesterase.

16. The viral vector of claim 1, wherein the cholinesterase is butyrylcholinesterase.

17. The viral vector of claim 16, wherein the plant codon-optimized DNA sequence encodes an amino acid sequence that is at least 90% identical to an amino acid sequence which corresponds to a human butyrylcholinesterase.

18. The viral vector of claim 17, wherein the plant codon-optimized DNA sequence encodes an amino acid sequence that is at least 95% identical to an amino acid sequence which corresponds to a human butyrylcholinesterase.

19. The viral vector of claim 18, wherein the plant codon-optimized DNA sequence encodes a human butyrylcholinesterase.

20. The viral vector of claim 1, wherein the vector contains a nucleic acid sequence at least 90% identical to a nucleic acid sequence of SEQ ID NOS: 1, 3, 5, 7, 9, 11, or 13.

21. The viral vector of claim 20, wherein the vector contains a nucleic acid sequence at least 95% identical to the nucleic acid sequence of SEQ ID NOS: 1, 3, 5, 7, 9, 11, or 13.

22. The viral vector of claim 21, wherein the vector contains the nucleic acid sequence of SEQ ID NOS: 1, 3, 5, 7, 9, 11, or 13.

23. The viral vector of claim 1, wherein the vector contains a nucleic acid sequence that encodes an amino acid sequence at least 90% identical to an amino acid sequence of SEQ ID NOS: 2, 4, 6, 8, 10, 12, or 14.

24. The viral vector of claim 23, wherein the vector contains a nucleic acid sequence that encodes an amino acid sequence at least 95% identical to an amino acid sequence of SEQ ID NOS: 2, 4, 6, 8, 10, 12, or 14.

25. The viral vector of claim 24, wherein the vector contains the nucleic acid sequence that encodes an amino acid sequence of SEQ ID NOS: 2, 4, 6, 8, 10, 12, or 14.

26. A method of transiently producing a cholinesterase using the vector of claim 1.

27-52. (canceled)
